(19) United States
(12) Patent Application Publication
(10) Pub. No.: US 2020/0129579 A1
(43) Pub. Date: Apr. 30, 2020

(54) METHOD FOR REPAIRING SKIN DAMAGE CAUSED BY PARTICULATE MATTERS (PMs)

(71) Applicant: Shanghai Zhongyi Daily Chemical Co., Ltd., Shanghai (CN)

(72) Inventors: Zhengzheng Liao, Shanghai (CN); Jing Nie, Shanghai (CN); Peiwen Sun, Shanghai (CN)

(21) Appl. No.: 16/662,431

(22) Filed: Oct. 24, 2019

(30) Foreign Application Priority Data
Oct. 26, 2018 (CN) 2018112591693
Oct. 26, 2018 (CN) 2018112606684

(51) Int. Cl. A61K 36/82; A61Q 19/10; C12Q 1/60; A61Q 19/08

(52) U.S. Cl. CPC A61K 36/82; A61Q 19/08; C12Q 1/60; A61Q 19/10

(57) ABSTRACT

The present invention provides the use of one or more active ingredients that can reduce the adverse effects of particulate matter (PM) on the skin, the active ingredients include polyphenolic substances. The active ingredient disclosed herein can significantly reduce the adverse effects of skin caused by PMs, and can be applied in many different aspects, such as skin care products with anti-pollution and anti-haze effect.

### CROSS REFERENCE TO RELATED APPLICATION

The present application claims the benefit of Chinese Patent Application No. 2018112591693, filed on Oct. 26, 2018; and Chinese Patent Application No. 2018112606684, filed on Oct. 26, 2018. The entireties of these applications including all tables, diagrams and claims are incorporated herein by reference.

### FIELD OF THE INVENTION

The present invention relates to biomarkers for evaluating the effects of PMs on the skin or hair, particularly, biomarkers and evaluation methods for the assessment of the impact of PM2.5 on skin or hair, and also relates to the substances or use of the substances which affect the skin stratum corneum (SC) cholesterol metabolism.

### BACKGROUND OF THE INVENTION

The following background is used to help the reader understand the present invention and there is no any scope limitation to this present invention.

With the global industrialization, atmospheric pollutants have caused serious human health problems. The World Health Organization identified four major air pollutants in its air quality guidelines, namely PM, ground ozone, nitrogen dioxide and sulfur dioxide. Of all these substances, PM2.5 is a tiny PM substance with an aerodynamic diameter of less than or equal to 2.5 μm. PM2.5, as the main component of air pollutants, impose significant threats to the cardiovascular system, respiratory system and skin. In addition, PM2.5 has a large surface area that absorbs chemical contaminants and metal ions. Studies have shown that long-term exposure to airborne PM activates aromatic hydrocarbon receptors (AhR) on the skin cell membranes, leading to skin aging, wrinkles and pigmentation. In addition, atmospheric pollutants can also induce and exacerbate the atopic dermatitis and other skin diseases.

The stratum corneum (SC) has a structure similar to bricks and mortar. “Bricks” refer to cornified keratinocytes, while “mortar” refers to the lipid components filled between them, including free fatty acids, ceramides and cholesterol. These three lipid components are stacked in a highly ordered three-dimensional structure, gluing the cells together to form a strong skin barrier. The function of the skin barrier depends on the integrity of the SC, especially the composition of lipids in it. Di Nardo A et al., have found that the skin barrier of patients with atopic dermatitis is impaired, with reduced level of ceramides, increased level of cholesterol, and lower ceramide/cholesterol ratios in SC.

### SUMMARY OF THE INVENTION

The first aspect of the present invention provides an application of the substances in the cholesterol metabolism pathway in the evaluation of the effects of PMs on the skin. In some embodiments, these substances are substances that directly affect cholesterol metabolism or indirectly affect cholesterol levels, participating in cholesterol synthesis and metabolism.

The so-called “participation” is that these substances can affect the synthesis or metabolism of cholesterol. These substances can be specific to genes. The level of transcription, translation, or the amount or activity of enzymes, substrates, precursors affected by gene expression, all of which are caused by PMs acting on the skin. In some ways, the metabolism of cholesterol occurs in whole or in part in the SC of the skin.

In some embodiments, these substances include enzymes in the cholesterol metabolism pathway, or direct products synthesized at stages or steps under the action of these enzymes, or one or both of the two. These direct products include, but are not limited to, cholesterol, squalene and other substances. The so-called metabolic pathway of direct products starting with cholesterol metabolism, in the action of the enzymes, the changes of the amount of some intermediate products, such as ATP Citrate Lyase, 3-Hydroxy-3-Methylglutaryl-CoA Reductase, Acyl-CoA Synthetase Short Chain Family Member 2, 3-Hydroxy-3-Methylglutaryl-CoA Synthase 1, Mevalonate Diphosphate Decarboxylase, Changes in direct substances such as acetate, citrate, acetyl-CoA, acetoacetyl-CoA, acetoacetyl-CoA, β-Hydroxy β-methylglutaryl-CoA, mevalonate, mevalonic acid-5-P, mevalonate-5-pyrophosphate, dimethylallyl pyrophosphate, farnesyl pyrophosphate, squalene, squalene-2,3epoxide, lanosterol, etc. can be used to indicate the damage of PMs to the skin.

In other embodiments, it can also be some in direct substances involved in the cholesterol metabolism pathway, such as the various enzymes listed above. The synthesis of these enzymes directly affects the synthesis or amount of upstream substrates. The enzymes can be one or more of the following: ATP Citrate Lyase, 3-Hydroxy-3-Methylglutaryl-CoA Reductase, Acyl-CoA Synthetase Short Chain Family Member 2, 3-Hydroxy-3-Methylglutaryl-CoA Synthase 1, Mevalonate Diphosphate Decarboxylase, Methylsterol Monooxygenase 1, Methylsterol Monooxygenase 1, Farnesyl-Diphosphate Farnesyltransferase 1, Squalene Epoxidase, Lanosterol Synthase, Low Density Lipoprotein Receptor, Stearoyl-CoA Desaturase, Fatty Acid Synthase.

In some embodiments, it can also be one or more of the direct products, or one or several of the indirect substances, or the combination of direct product and indirect substances, to assess the effect of PMs on the skin SC. Cholesterol metabolism pathways are common knowledge, but existing technology does not draw connection between cholesterol metabolism and PM, and the present invention found that the metabolic pathway of cholesterol was directly affected by PMs. In some ways, cholesterol or squalene in the metabolic pathway of cholesterol is used to assess the effects of PM on the SC.

The second aspect of the present invention provides a method for evaluating the effect of the active ingredient on mitigating the impact of PM from the atmosphere, e.g. PM2.5, on the skin; or provides a screening method for active ingredients which mitigate the impact of PM from the atmosphere, e.g. PM2.5, on the skin. In some embodiments, the evaluation or screening of the impact of PM from the atmosphere, e.g. PM2.5, on the skin is provided. Here, the so-called impact generally refers to harmful or damage effects of PMs from the atmosphere, which are detrimental or damage to skin. This adverse effect is caused by PMs interfering with the metabolic pathway of cholesterol in the SC of the skin.

In some embodiments, the evaluation or screening of active ingredients is to see what the effects of these so-called active ingredients on the skin are, such as exacerbating, worsening the adverse effect, or having no effect, or having the effect of improving and repairing the damage caused by harmful PMs, and helping the development of normal skin.

In some embodiments, the active ingredient has an intervention effect on the adverse effects of PMs, which refers to preventing the exacerbation of skin damage and the improvement, repair or treatment of skin damage.

In some embodiments, the method here includes having the active ingredient to be tested to treat the skin, and if the relevant substance in the cholesterol metabolism pathway changes in the skin, the effect of the active ingredient measured is judged by the change. In some ways, the effects include making the skin more damaged or improved post the impact of PM, such as PM2.5, resulting in damage reduction, or damage repair. On the other hand, changes in related substances include an increase in some substances or a decrease in some substances associated with or directly related to the effects of the injury. In some ways, for example, an increase in cholesterol indicates that the active ingredient does not improve the skin with regard to the damage of PM, such as PM2.5. Otherwise it has a good effect on the skin. Also for example, an increase in squalene indicates that the active ingredient has a positive effect on the skin with regard to the damage of PM, such as PM2.5. Otherwise it has no improvement. That is, it may not work or do the opposite. By this method, some active ingredients can be effectively screened, which can reverse the effect of PM on cholesterol metabolism pathway. When there is no additional active ingredient, the PMs can change the course of cholesterol metabolism, eventually increasing cholesterol to higher than normal level. When an additional active ingredient is applied, it can change cholesterol metabolism pathway, eventually return cholesterol to normal levels, such substances are effective ingredients. Of course, substances that do not affect the direction of the synthetic pathway of cholesterol can be considered ineffective active ingredients. Optionally, one can also identify or screen out a class of substances; such substances act on skin, changing or affecting the metabolic pathway of cholesterol, and ultimately increase cholesterol. Such substances are harmful substances.

In some embodiments, when, before and after PMs act on the skin, including one or more of skin tissues, skin cells, SC cells, SC tissues, or three-dimensional model skin, or in the action process, let active ingredients act on skin, skin tissues, skin cells, SC cells, SC tissues, or three-dimensional models and detect the changes in cholesterol metabolism-related substances, to screen effective, ineffective or harmful active ingredients according to the changes. In some embodiments, effective active ingredients are ultimately available and can be used to alleviate adverse effects of PMs on the skin. In some embodiments, the skin tissues, skin cells, SC cells and SC tissues are in vitro tissues or cells, of course, optionally, can be non-in vitro skin tissue, skin cells, SC cells and SC tissues.

In a third aspect of the present invention, the present invention provides the use of active ingredients in preparing reagents for alleviating damage of atmospheric PMs to the skin. In some embodiments, active ingredients may be used in personal care reagents that have anti-pollution, anti-haze effects, such as skin care products, clean bath products, or some care products.

These active ingredients may be obtained by screening through the foregoing methods. These active ingredients include, but are not limited to, polyphenols. In some embodiments, these polyphenols can be any polyphenol, such as polyphenols extracted from plants, animals or microbes. In some embodiments, polyphenols down-regulate HMGCS1, LDLR, and FASN in the cholesterol synthesis pathway, causing the final cholesterol level to tend to stabilize.

Although it has been found in the prior art that polyphenols have anti-inflammatory and anti-antioxidant effects, no one has found that polyphenols can improve the effect of micro-particles on the skin, especially without changing the cholesterol metabolism pathway.

Therefore, in a fourth aspect, the present invention provides the uses of polyphenols in preparing reagents that can improve the adverse effects of PMs on the skin. In some embodiments, the polyphenols are derived from plants, microbial fermentation products, mammals. In some preferred embodiments, the green plant is Camellia sinensis. In some preferred embodiments, the polyphenols are tea polyphenols. In some embodiments, the PMs are atmospheric PMs, for example, PM2.5. In some embodiments, the adverse effects of PMs on the skin include the effect of PMs on the skin SC. In some embodiments, the effect of PMs on the skin SC includes the effect of PMs on cholesterol metabolism-related substances in SC.

In some embodiments, the cholesterol metabolism-related substances include genes involved in the regulation of cholesterol metabolism. In some embodiments, the genes include one or more genes of ACLY, ACSS2, HMGCR, HMGCS1, MVD, MSMD1, FDFT1, SQLE, LSS, LDLR or SCD, FASN, INSIGL. Preferably, the genes are HMGCS1, LDLR and FASN.

In some embodiments, the cholesterol metabolism-related substances include enzymes involved in the cholesterol metabolism pathway. In some embodiments, the enzymes include one or more of ATP Citrate Lyase, 3-Hydroxy-3-Methylglutaryl-CoA Reductase, Acyl-CoA Synthetase Short Chain Family Member 2, 3-Hydroxy-3-Methylglutaryl-CoA Synthase 1, Mevalonate Diphosphate Decarboxylase, Methylsterol Monooxygenase 1, Methylsterol Monooxygenase 1, Farnesyl-Diphosphate Farnesyltransferase 1, Squalene Epoxidase, Lanosterol Synthase, Low Density Lipoprotein Receptor, Stearoyl-CoA Desaturase, Fatty Acid Synthase.

In some embodiments, the cholesterol metabolism-related substances include some substrates or precursors in the cholesterol synthesis initial stage or synthetic steps. In some embodiments, the substrates or precursors include one or more of acetate, citrate, acetyl-CoA, acetoacetyl-CoA, acetoacetyl-CoA, β-Hydroxy β-methylglutaryl-CoA, mevalonate, mevalonic acid-5-P, mevalonate-5-pyrophosphate, dimethylallyl pyrophosphate, farnesyl pyrophosphate, squalene, squalene-2,3epoxide, lanosterol. In some embodiments, the cholesterol metabolism-related substance is cholesterol.

The effective substances screened by the foregoing method can be used for various purposes, for example, applied to the skin surface to alleviate adverse effects of PMs on the skin. These substances and active ingredients can be used as some specific forms to prepare cosmetic reagents, to improve, prevent, and reverse the adverse effects of PMs on the skin, thereby repairing the barrier, so that the skin can be protected, to avoid or mitigate the hazards of PMs. In some preferred embodiments, wherein the agent is a skin care preparation, and the skin care agents include substances screened by the foregoing methods, such as polyphenols. Sin care preparations may be in any form, and may be one of the solutions, water agents, suspensions, masks, lotions, creams, pastes, gels, dry powders, wet powders, sprays. Of course, these agents can be mammalian or human care product agents such as skin care, cleansing, beauty, bathing, and toiletries.

In addition to cholesterol metabolism-related genes, we firstly discovered that genes that have not been previously reported or are directly or indirectly related to PM2.5 further include S100A8, S100A9, Krt6b, TXNRD1, FGFBP1, MT2A, CD9, AREG, ITGB1, LAMB3, LAMA3.

Therefore, in a fifth aspect of the invention, S100A8, S100A9, Krt6b, TXNRD1, FGFBP1, MT2A, CD9, AREG, ITGB1, LAMB3 or LAMA3 genes are up-regulated in PM2.5-stimulated keratinocytes. That is, the up-regulation of expressions of these genes leads to some adverse effects under the action of PM2.5, for example the following adverse effects of up-regulation of these genes. Conversely, as described above, if some active ingredients applied can reverse, improve or repair the adverse effects caused by up-regulation of these genes, the substances have corresponding functions, and such adverse effects are directly caused by PMs in the atmosphere, for example, PM2.5. For example, when some substances are applied to skin, for example, to the SC cells, it is found that S100A8, S100A9 gene expression is not up-regulated, for example, down-regulation of the latter does not change, indicating that the substance can improve or repair or treat specific dermatitis, which is directly caused by PM2.5.

S100A8 and S100A9 belong to S100 protein family and are distributed in the granular layer, the spinous layer and the basal layer. Their main role is to involve in the host defense process related to NADPH oxidase activation. Studies have shown that the two S100 proteins play an important role in epidermal wound repair, differentiation and stress response, and their expression levels are extremely low in normal epidermis, but are very high in the skin of psoriasis patients, and their expression levels are proven to be up-regulated in the skin of specific dermatitis. Krt6b belongs to the keratin family and can characterize the keratinocyte division rate, which is a typical marker of wound healing, and is up-regulated in skin diseases such as specific dermatitis and ichthyosis, etc. TXNRD1 encodes a thioredoxin reductase and is a typical antioxidant gene, which is regulated by Nrf2 transcription factor. FGFBP1 encodes a fibroblast growth factor binding protein that is involved in the regulation of skin cell division and is associated with wound healing and angiogenesis. MT2A is a metal-binding protein that can promote cell proliferation, and it has been found that its expression level is associated with the formation of skin scar. CD9 is a cell surface protein that is involved in a variety of biological processes, such as wound healing, by coupling the signaling of intracellular signals. AREG amphiregulin functions in inflammatory epidermal hyperplasia and sebaceous gland enlargement, and it has been reported that its expression in human airway epithelial cells is up-regulated under the influence of PM2.5. LAM laminin is involved in wound healing and host defense. ITG integrin mediates adhesion of skin cells and is closely related to wound healing and inflammatory response. It is stimulated by mechanical stress and external damage to maintain the skin homeostasis.

In a sixth aspect, the present invention provides a method of improving skin comprising contacting polyphenols with the skin, wherein the skin is affected by PMs. In some preferred embodiments, the polyphenols are derived from plants, microbial fermentation products, mammals. In some preferred embodiments, the green plant is Camellia sinensis. In some preferred embodiments, wherein the polyphenols are tea polyphenols. In some preferred embodiments, the PMs are PM2.5. In some preferred embodiments, the effect of the PMs on the skin includes the effect of PMs on the skin SC. In some preferred embodiments, the effect of the PMs on skin SC includes the effect of PMs on cholesterol metabolism-related substances in SC. In some preferred embodiments, wherein the cholesterol metabolism-related substances include genes involved in the regulation of cholesterol metabolism. In some preferred embodiments, wherein the cholesterol metabolism-related substances include enzymes involved in the cholesterol metabolism pathway. In some preferred embodiments, the cholesterol metabolism-related substances include some substrates or precursors in the cholesterol synthesis initial stage or synthetic steps. In some preferred embodiments, wherein the gene comprises one or more genes of ACLY, ACSS2, HMGCR, HMGCS1, MVD, MSMD1, FDFT1, SQLE, LSS, LDLR or SCD, FASN, INSIGL. In some preferred embodiments, wherein the gene is HMGCS1, LDLR or FASN. In some preferred embodiments, wherein the enzyme comprises one or more of ATP Citrate Lyase, 3-Hydroxy-3-Methylglutaryl-CoA Reductase, Acyl-CoA Synthetase Short Chain Family Member 2, 3-Hydroxy-3-Methylglutaryl-CoA Synthase 1, Mevalonate Diphosphate Decarboxylase, Methylsterol Monooxygenase 1, Methylsterol Monooxygenase 1, Farnesyl-Diphosphate Farnesyltransferase 1, Squalene Epoxidase, Lanosterol Synthase, Low Density Lipoprotein Receptor, Stearoyl-CoA Desaturase, Fatty Acid Synthase. In some preferred embodiments, wherein, substrates or precursors include one or more of acetate, citrate, acetyl-CoA, acetoacetyl-CoA, acetoacetyl-CoA, β-Hydroxy β-methylglutaryl-CoA, mevalonate, mevalonic acid-5-P, mevalonate-5-pyrophosphate, dimethylallyl pyrophosphate, farnesyl pyrophosphate, squalene, squalene-2,3epoxide, lanosterol. In some preferred embodiments, the cholesterol metabolism-related substance is cholesterol. In some preferred embodiments, wherein the PMs are atmospheric PMs.

Additionally, the present invention provides a method of repairing skin SC, comprising contacting polyphenols with the skin, wherein the SC is damaged by the PMs. In some embodiments, the PM is PM2.5. In some preferred embodiments, wherein the damage to the SC is the alteration of metabolism of cholesterol metabolism-related substances in the SC. In some preferred embodiments, wherein the metabolism-related substance is cholesterol.

### Beneficial Effect

The present invention proves for the first time that the metabolism of cholesterol in the skin is directly related to PMs and is directly affected by airborne PMs, for example PM2.5. It can affect the cholesterol level in the SC which is directly evidenced at the genetic and cellular levels. This mechanism allows the screening of active ingredients that alleviate the adverse effects of PMs on the skin, and the active ingredients can protect against the adverse effects of PMs, which has been demonstrated by the genetic levels, final cholesterol and squalene levels.

### BRIEF DESCRIPTION OF THE DRAWINGS

FIGS. 1A and 1B show the MTT test results of keratinocytes. FIGS. 1A and 1B show that the PM2.5 concentration depends on decreased cell activity and cell morphological abnormalities, with the manifestation of cell shrinking, rounding and reduced adherent cells. FIG. 1A shows the effect on cell activity, FIG. 1B shows the effect on cell morphology, indicating that PM2.5 has a significant damage to skin cells.

FIG. 2 is a sorted chart of up-regulated genes after stimulated by PM2.5 according to the degree of enrichment, among the first 20 GO entries and pathway charts, the cholesterol metabolism is most prominent.

FIG. 3 is a graph showing the degree of up-regulation of up-regulated genes in the cholesterol hormone pathway caused by PM2.5 stimulation, wherein a graph of up-regulated gene transcription levels of PM2.5 treatment vs control treatment.

FIG. 4 shows that the GTE+PM2.5 group reverses the trend of expression and down-regulates cholesterol metabolism-related genes and the degree of down-regulation compared to the cholesterol metabolism-related genes up-regulated by PM2.5 stimulation group.

FIGS. 5A and 5B show the effects of P2.5 and PM2.5+GTE on cholesterol and squalene contents at different times in the 3D skin models. FIG. 5A is a graph showing changes in cholesterol and squalene contents after treated by PM2.5 at different times; FIG. 5B is graph showing the changes in cholesterol level after treated by PM2.5+GTE, and PM2.5 respectively.

FIG. 6 is a graph showing the substance and multiple enzyme genes in the cholesterol metabolism pathway and the corresponding synthetic steps.

### DETAILED DESCRIPTION

The structure involved in the present invention or the technical terms used therein will be further described below. The illustrations are merely illustrative of how the manner of the invention can be implemented and are not intended to limit the invention in any way.

### Detection

Detection is to test the presence of a substance or material, for example, but not limited to, chemicals, organic compounds, inorganic compounds, metabolites, drugs or drug metabolites, organic tissues or metabolites thereof, nucleic acids, proteins or polymers. In addition, the detection is to test the number of substances or materials.

### Relationship Between Cholesterol Metabolism Pathway and PMs

Skin SC plays an important barrier function in the external environment. It is the structure that is in direct contact with pollutants in the environment firstly. The toxic substances adsorbed by PMs will damage the normal barrier function of the skin. The skin barrier is composed of keratinocytes and lipid components between keratinocytes. Cholesterol, ceramide, and free fatty acids are the main barrier lipids; their ratio and type determine the normal functions of barrier. It has been demonstrated that the acute barrier damage caused by tape tearing or acetone will affect the cholesterol level in the SC.

The team members of the present invention are surprised to find that atmospheric PM, such as PM2.5, has an important influence on the metabolic pathway of cholesterol. Some genes involved in the cholesterol metabolism pathway have significantly increased expression levels relative to the normal state, while some genes have significantly decreased expression levels. These genes may have direct impact on the content or activity of enzymes, substrates or intermediates that cause the cholesterol metabolism pathway. Of course, genes may not have a direct correspondence but an indirect relationship with these enzymes, substrates or intermediates. For example, genes directly affect the amount or activity of enzymes, and the enzymes indirectly affect the synthesis of intermediates or precursors related to the cholesterol metabolism.

Another surprising finding is that atmospheric PM, for example PM2.5, can directly affect some of the related substances in the cholesterol metabolism pathway, thereby increasing or decreasing the expression of genes in the cholesterol metabolism pathway or enzymes affecting these substances. As a result, the level of cholesterol is finally increased relative to normal level. Still another surprising finding is that the atmospheric PMs, for example PM2.5, increase the level of cholesterol in the epidermis, while the level of squalene, the precursor for cholesterol synthesis is decreased.

This is contrary to some conventional understandings in the prior art. Although studies have shown that lipid components can be used to measure barrier integrity, there is no definitive conclusion about the effects of air pollutants, especially PMs, on cholesterol and its associated metabolites in SC. A series of clinical trials conducted worldwide from 1999 to 2014 have showed that residents living in areas with severe air pollution have high levels of sebum oxidation and lower SC integrity. However, study results in Mexico and Shanghai revealed that people living in heavily polluted areas have higher levels of sebum and a lower squalene/cholesterol ratio, but the cholesterol level has no significant change. This indicates that, atmospheric PMs or PMs formed by atmospheric pollution seem to have no direct effect on the lipid composition in SC, especially the final product cholesterol. However, in the present invention, in fact, atmospheric PMs or PMs formed by atmospheric pollution may directly affect various stages of cholesterol synthesis route, thereby ultimately interfering with cholesterol synthesis and increasing its relative content (relative to the normal content) in SC.

In the present invention, using a representative substance in atmospheric PM and PM2.5-treated primary human epidermal keratinocytes (pHEK), the effect of PM2.5 on the skin barrier is analyzed based on the obtained transcriptomics results. It is found that numerous up-regulated genes are associated with cholesterol metabolism. The transcriptomics results allow us to focus on changes in expressions of cholesterol metabolism-related genes. Based on the changes in the transcriptional levels of these genes, a PM2.5 treatment model is constructed using in vitro three-dimensional epidermal tissue (3D-ETM) to conduct experiments and investigate the effect of PM2.5 on the contents of cholesterol and squalene, the cholesterol precursor. A correlation analysis is performed from gene expression changes and cholesterol metabolic routes, and it is found that the transcription of these altered genes is closely related to the cholesterol metabolic route, therefore, it is believed that the atmospheric PMs affect the cholesterol metabolic route in the epidermal tissues, or the cholesterol metabolic route in the SC, thereby eventually leading to an increased cholesterol level and a decreased level of cholesterol precurs or squalene.

In addition, it is found that the up-regulation of some genes that are not directly related to cholesterol metabolism is directly caused by atmospheric PMs, such as PM2.5. These genes are also our new findings.

### Biomarkers

The present invention provides some biomarkers, which can be used to evaluate or assess the effect of PMs on the skin, especially the effects of PMs on SC, more particularly, to evaluate or assess the effect of PMs on cholesterol metabolism in SC.

In some embodiments, PMs may be PMs in the atmosphere at anytime, anywhere, or PMs in a specific environment. In some embodiments, these biomarkers include cholesterol metabolism-related substances. In some embodiments, these biomarkers include direct or indirect cholesterol metabolism-related substances. In some embodiments, these biomarkers include directly related substances or/and indirectly related substances on the cholesterol metabolism pathway.

The present invention provides the use of cholesterol metabolism-related biomarkers in assessing the effects of PMs on the skin, in particular, the use in evaluating or assessing the effect of PMs on the SC, and more specifically, the use in evaluating or assessing the effect of PMs on the synthesis of cholesterol in the SC.

Among the uses, these biomarkers can be used to evaluate the extent of effect of PMs or changes in the specific content of PMs on the skin in different regions or at different periods of time.

The PMs herein may be PM2.5, or PMs formed by different chemical elements or mixtures of different chemical substances. In addition, this direct association has been established to evaluate or screen some active ingredients to reverse, improve or prevent some adverse effects of PMs on the skin. Of course, it will be understood that these biomarkers can be used to evaluate or measure the extent of active ingredients on reversing, improving or preventing some adverse effects of PMs on the skin. It will be understood that these biomarkers can be used to evaluate the negative effects of some active ingredients on the skin adverse process caused by PMs, thereby these biomarkers are not used to act on the skin. It will be described in detail below.

In some embodiments, these biomarkers include genes at the molecular level orenzymes, substances, or nucleic acids or genes involved in cholesterol metabolism pathway, or the amount or activity of enzymes involved in the cholesterol metabolism pathway, or some substrates or precursors in the cholesterol synthesis initial stage or synthetic steps, alternatively, a combination of one or more of the above at the genetic level, the enzyme level, or substrate precursors. In some other embodiments, these biomarkers are not substances directly related to cholesterol metabolism but other substances, for example, S100A8, S100A9, Krt6b, TXNRD1, FGFBP1, MT2A, CD9, AREG, ITGB1, LAMB3, LAMA3 and other genetic materials.

The “cholesterol metabolism pathway” herein may also include physiological processes such as synthesis or decomposition of cholesterol, transportation, etc. The substances related to the metabolic process are directly or indirectly involved in the process.

The effect of PMs on the skin is indicated by the expression of these genes or by changes in the transcription level, for example, increase or amplitude of increase, decrease or amplitude of decrease. For example, the adverse effect of PMs on the skin can be indicated by the reduction or degree of reduction of some genes at the level of transcription. On the contrary, it indicates the extent of improvement and reversal of skin by PMs. In some embodiments, the gene markers at the molecular level include one or more genes of ACLY, ACSS2, HMGCR, HMGCS1, MVD, MSMD1, FDFT1, SQLE, LSS, LDLR or SCD, FASN, INSIGL. The elevated levels of transcription of these genes indicate the increase in the adverse effects of PMs on the skin; conversely, a decrease in the transcriptional level of these genes indicates that the adverse effects of the PMs on the skin are improved or reversed. For example, some active ingredients act on the skin and some genes are found to be down-regulated at the transcriptional level (relative to the effect of PMs alone), indicating that these active ingredients have the effect of improving or reversing the adverse effects, and the degree of down-regulation indicates the extent of improving or reversing adverse effects by the active ingredients.

In some embodiments, biomarkers include enzymes that directly affect the various steps in the cholesterol pathway, causing a series of biological reactions that ultimately affect the cholesterol level (relative to normal health state). In some embodiments, these enzymes include one or more of ATP Citrate Lyase, 3-Hydroxy-3-Methylglutaryl-CoA Reductase, Acyl-CoA Synthetase Short Chain Family Member 2, 3-Hydroxy-3-Methylglutaryl-CoA Synthase 1, Mevalonate Diphosphate Decarboxylase, Methylsterol Monooxygenase 1, Methylsterol Monooxygenase 1, Farnesyl-Diphosphate Farnesyltransferase 1, Squalene Epoxidase, Lanosterol Synthase, Low Density Lipoprotein Receptor, Stearoyl-CoA Desaturase, Fatty Acid Synthase. The effect of PMs on the skin, or the interference or improvement of skin by active ingredients is assessed by the amount or activity, or amount and activity of these enzymes.

In some embodiments, these biomarkers may be substrates and precursors in the cholesterol metabolism pathway. These biomarkers include, for example, one or more of acetate, citrate, acetyl-CoA, acetoacetyl-CoA, acetoacetyl-CoA, β-Hydroxy β-methylglutaryl-CoA, mevalonate, mevalonic acid-5-P, mevalonate-5-pyrophosphate, dimethylallyl pyrophosphate, farnesyl pyrophosphate, squalene, squalene-2,3epoxide, lanosterol.

The term “effect” herein refers to a bad or negative effect produced when PMs act on the skin. Generally, this effect may change the activity or content of various enzymes in the synthetic route, or the level of transcription of genes associated with the enzymes, which ultimately leads to a cholesterol level higher than normal level, and thereby causing a change in the ratio of skin SC lipid components and abnormal barrier functions. This effect actually refers to effect on skin damage. For active ingredients, the effect of this active ingredient on the skin may be to reverse the adverse effects of PMs on the skin or to worsen or intensify the adverse effects of PMs on the skin; of course, optionally, these active ingredients may not have any function. At the molecular level, these active ingredients generally cause down-regulation or up-regulation of some genes, or down-regulation or up-regulation of a plurality of genes, which ultimately presents different effects. For example, effective active ingredients can improve or reverse the adverse effects, while harmful active ingredients can accelerate and worsen such adverse effects; ineffective active ingredients have no effect on the skin's adverse effects caused by PMs.

By using this method, it is possible to evaluate the damage or degree of damage to the skin by atmospheric PMs in different places. The adverse or abnormal direction herein is relative to a healthy and normal standard. In addition, when additional active ingredients are applied to the skin, these additional substances can be evaluated whether or not to alleviate or reverse the adverse effects of the PMs on the skin. These additional substances be active ingredients such as Camellia sinensis extract, or polyphenols, of course, additional active ingredients may also be a type of substances for reducing the adverse effect of PMs on the skin. When these substances act on the skin, they can improve this effect or eliminate the adverse effects of PMs on the skin, or control the damage of PMs to the skin, or reduce or slow down the damage of atmospheric PMs to the skin, especially the effect on cholesterol level in the SC.

### PMs

The “PMs” herein generally refers to atmospheric particulate matters, which are general term for various solid and liquid particulate materials present in the atmosphere. The various particulate materials are uniformly dispersed in the air to form a relatively stable and bulky suspension system, that is, an aerosol system. Therefore, atmospheric particulate matters are also called atmospheric aerosols (Hinds, wC Aerosol Technology: Properties, Behavior, And Measurement of Airborne Particles [M], Wiley, 1999, New York). Fine PMs are also known as fine particles, PM2.5. Fine PMs refer to particulate matter (PM2.5) with an aerodynamic equivalent diameter of 2.5 microns or less in ambient air. Compared with larger atmospheric particulate matters, PM2.5 has small particle size, large area, strong activity, is easy to attach toxic and harmful substances (such as heavy metals, microorganisms, etc.), and has a long residence time and a long transport distance in the atmosphere. Therefore, it has a greater impact on human health and quality of atmospheric environment.

Of course, in addition to the PMs in the atmosphere, there are also PMs in special environments, such as PMs in local range, for example, PMs in some specific plant environments may have effect on the skin.

### Skin or Hair

The scalp has a structure similar to the skin and consists of the epidermis, the dermis and the subcutaneous tissue. Keratinocytes form the epidermis, the dermis is a dense network of collagen fibers, with elastic blood vessels and lymphatic vessels, immune cells, hair follicles, nerve fibers and glands. There are approximately 300 sweat glands and 600 hairs per square centimeter in the healthy scalp, with a maximum of 5 sebaceous glands in each hair follicle. Studies have shown that air pollution mainly damages the scalp rather than the hair, which can cause hair loss or damage the skin barrier to make the scalp to become sensitive.

### Methods of Evaluation

The invention provides a method for evaluating the effects of PMs on the skin by biomarkers. In some embodiments, one or more of these biomarkers are used to evaluate the effects of the PMs on SC. In some embodiments, one or more of these biomarkers are used to evaluate the effects of PMs on the cholesterol metabolism pathway in the SC.

In some embodiments, the method comprises allowing PMs to act on the skin, determining changes in the amount of these biomarkers, to directly associate the PMs with the cholesterol metabolism pathway and evaluate the effect of PMs on the skin. More specifically, elevated levels or elevated activity of some biomarkers indicate an increase in the adverse effects of PMs on the skin, and the decreased levels or decreased activity of some biomarkers indicate a decrease in the adverse effects of PMs on the skin. The skin herein may be one or more of skin tissues, skin cells, SC cells, SC tissues, or three-dimensional model skin. In some embodiments, when, before and after PMs act on the skin, or in the action process, let active ingredients act on skin, skin tissues, skin cells, SC cells, SC tissues, or three-dimensional models and detect the changes in cholesterol metabolism-related substances, to screen effective, ineffective or harmful active ingredients according to the changes.

In some embodiments, effective active ingredients are ultimately available and can be used to alleviate adverse effects of PMs on the skin. In some embodiments, the skin tissues, skin cells, SC cells and SC tissues are in vitro tissues or cells, of course, optionally, can be non-in vitro skin tissue, skin cells, SC cells and SC tissues.

In some embodiments, these biomarkers include genes at the molecular level or changes in nucleic acids or genes of enzymes, substances, receptors in the cholesterol metabolism pathway, may also include the amount or activity of enzymes involved in the cholesterol metabolism pathway, or some substrates or precursors in the cholesterol synthesis initial stage or synthetic steps, alternatively, a combination of one or more of the above at the genetic level, the enzyme level, or substrate precursors.

In some embodiments, an elevated level of cholesterol or a decreased level of squalene in the SC indicates an increase in the adverse effects of PMs on the skin. Conversely, if the level or activity of some biomarkers is not or is substantially not changed, it indicates that PMs have no adverse effects on the skin.

PMs herein refer to any type of PMs at anytime, anywhere, which can be harmful, or harmless or unknown PMs, or PMs to be detected. According to the method of the present invention, the adverse effect of PMs on the skin barrier can be detected or evaluated, and the effect of unknown PMs can be evaluated by detecting the substances related to the cholesterol metabolism pathway.

### Screening Methods and Active Ingredients

The invention provides a method for screening the effect of an active ingredient on the skin because of the interference of PMs. The active ingredients herein are different from the enzymes, substances or precursors in the cholesterol pathway, and are not PMs, but they are some substances that may be used to improve and reverse the adverse effects of PM on the skin; of course, they may be a type of substances that may worsen or accelerate the adverse effects of PMs on the skin. In some methods or uses, the aforementioned biomarkers are used as indicators to screen such active ingredients, thereby improving and reversing the adverse effect of PMs on the skin. In some methods, before, when and after PMs act on the skin or in the process of action, active ingredients to be tested are applied. If the content or activity of the biomarkers (cholesterol metabolism-related substances) changes, the biomarkers are associated with the active ingredients to be tested, thereby obtaining a type of substances that can improve, reverse the adverse effects of PMs on the skin or can worsen and accelerate the adverse effects of PMs on the skin. In some methods, when the cholesterol level is increased (relative to normal level), it indicates that the active ingredient has no beneficial effect. Conversely, if the cholesterol level is reduced or not increased, it indicates that the active ingredients have an advantageous effect, for example, improving or reversing the adverse effects of PMs. One of ordinary skill in the art will know that biomarkers in the cholesterol metabolism pathway can be detected by such methods, and the changes in activity or contents and the effect of active ingredients are readily available. The “association” herein means that the action of active ingredients is directly related to changes in substances associated with the cholesterol metabolism pathway, and through the changes, effective, ineffective or harmful active ingredients can be obtained. For example, when active ingredients are added to make the cholesterol level to normal, the active ingredients are considered to be effective substances. This relationship can be positive or negative, of course, it may be unrelated.

The skin herein may be one or more of skin tissues, skin cells, SC cells, SC tissues, or three-dimensional model skin. In some embodiments, when, before and after PMs act on the skin, or in the action process, let active ingredients act on skin, skin tissues, skin cells, SC cells, SC tissues, or three-dimensional models and detect the changes in cholesterol metabolism-related substances, to screen effective, ineffective or harmful active ingredients according to the changes. In some embodiments, the skin tissues, skin cells, SC cells, and SC tissues are in vitro tissues or cells, and of course, optionally, non-in vitro skin tissues, skin cells, SC cells, and SC tissues.

We investigate the intervention of some active ingredients (for example, plant-derived ingredients) on PM2.5-induced skin damage using the above method. In some embodiments, Camellia Sinensis is a traditional economic plant that can be processed by varying degrees of fermentation. Green tea is made from fresh tea leaves and undergoes a fine drying step to avoid oxidation and polymerization of phenols. Epigallocatechingallate (EGCG), a monomeric flavanol, is found to be the major polyphenol in green tea.

Tea polyphenols are known to have potent anti-inflammatory and anti-oxidation effects in vitro and in vivo. Studies have shown that tea polyphenols can reduce skin edema and erythema caused by UV rays, and protect DNA from damage by UV. In this study, we have found that the role of polyphenol-rich tea polyphenols is to reverse the effect of PM2.5 on skin gene expression at the transcriptome level, and we have validated the change in key lipid biomarkers in 3D-ETM by liquid chromatography-mass spectrometry (LC-MS).

Therefore, another important finding of the present invention is that polyphenols can reverse the effect of PM2.5 on skin gene expressions, and finally under the effect of PMs, the cholesterol synthesis in SC is at a normal level or its precursor and squalene are also at a normal level. That is to say, these polyphenols can repair the negative effects of PMs on SC (for example, reducing the content of cholesterol in the SC), such that the cholesterol in the SC maintains normal levels. On the other hand, these polyphenols can prevent or prevent the negative effects of PMs on SC (for example, maintaining the normal content of cholesterol in SC), so that the cholesterol in SC is maintained at a normal level without being affected by PMs in the external air pollution. Thus, polyphenols can reverse, repair, and prevent the adverse effects of cholesterol in SC, allowing it to be at a normal level, for example, lowering the cholesterol level when elevated, or preventing possible or potential increase of cholesterol. Moreover, in terms of the mechanism, the present invention also demonstrates that tea polyphenols down-regulate the transcription levels of HMGCS1, LDLR and FASN genes in the cholesterol metabolism pathway (these genes are up regulated under the action of PM2.5), thereby ultimately allowing cholesterol to be maintained at a relatively normal level in the SC. Polyphenols are actually a general term for a type of mixtures. The content of polyphenols from different sources may be varied, but the overall mechanism of action has no substantial difference. Therefore, it will be readily understood by one of ordinary skill in the art that any polyphenol has the similar function of the tea polyphenols of the present invention when reading the specification of this patent.

The polyphenols herein may be plant-derived polyphenols, animal-derived polyphenols, or polyphenols from microbial fermentation products.

In some preferred embodiments, polyphenols herein include those from other plants in addition to those from Camellia sinensis. In another embodiment, polyphenols are extracts from green plants, for example, plant polyphenol. A plant polyphenol is a kind of secondary metabolite with polyphenolic structure that are widely distributed in plants, mainly in the skin, roots, leaves and fruits of plants. In the narrow sense, a plant polyphenol is tannins or tannic acids, and its relative molecular mass is between 500 and 3000; in a broad sense, it further includes small molecular phenolic compounds such as anthocyanins, catechins, quercetin, gallic acid, ellagic acid, arbutin and other natural phenols. Tea polyphenols (TP), also known as tea tannins, is a generic term for a class of polyhydroxy compounds contained in Camellia sinensis, accounting for about 20% of the dry weight of Camellia sinensis. Its main component is catechins, accounting for about 80% of its total amount. In addition, it also contains flavanols, flavanones, phenolic acids and anthocyanins and their aglycones. Among various Camellia sinensis, green tea has the highest content of tea polyphenols, about 15% to 25% of the dry weight of Camellia sinensis, followed by oolong tea and black tea. In addition to tea polyphenols, it can be any polyphenols such as apple polyphenols, polyphenols of grapes and grape wines, polyphenols in beer, and polyphenols in plant polyphenol vegetables.

Apple polyphenols are a general term for polyphenols in apples, including anthocyanins, flavanols, phenolic acids, and catechins, etc. In some apple varieties used for wine making, its content can reach 7 g/kg (by fresh weight), while its content in common fresh foods is within the range of 0.5-2 g/kg [3]. According to the survey of nutritionists in the Netherlands and the United States, apples are the third largest dietary source of phenolic substances after teas and onions.

Grapes are rich in polyphenols. It is mainly distributed in fruit stalks, peels and kernels, while the content in kernels is highest, 3% to 7% [4]. Among more than 1,000 substances detected in wine, a variety of polyphenols, including anthocyanidins, flavonoids, phenolic acids and resveratrols are contained. The polyphenol content in wine is determined by potassium permanganate method, which is 1˜3 g/L on average. In addition, the astringency and bitterness of red wine are mostly derived from phenolic substances, and the ruby color of red wine is also closely related to the content of polyphenols.

There are many kinds of polyphenols in beer, including flavanols, flavonols, anthocyanins and phenolic acids [5], among which, more than 50 kinds of proanthocyanidins are detected. About 80% of the polyphenols in beer come from barley, and about 20% come from hops. The content of polyphenols in malt is about 0.1% to 0.3%, mainly in the husk and aleurone layer, and little in the endosperm. Polyphenols in lupulus mainly exist in the anterior leaves and lupulin of lupulus, accounting for 2% to 4% of dry weight. Polyphenols are closely related to the quality of beer, and their content has an important influence on the non-biological stability, taste, foam and color of beers.

Plant polyphenol is also widely distributed in vegetables, such as day lily, spinach, lotus root, colorful purple cabbage, red spinach, purple radish that are commonly taken by people. The traditional vegetable artichokes in southern Europe are also rich in polyphenolic compounds such as cynarin and flavonoids, etc.

One of ordinary skill in the art will appreciate that any polyphenols have the effect of the present invention, which can improve the adverse effects of PMs on the skin. It is also a novel use of polyphenols discovered in the present invention.

### Detailed Description of the Embodiments

The present invention illustrates how the invention is implemented by way of examples. These examples are merely a limited list under the essence of the present invention and are not intended to limit the scope of the invention.

### Example 1: Collection and Analysis of PM2.5 Samples

PM2.5 samples were provided by Institute of Earth Environment of the Chinese Academy of Sciences (Xi'an) that were collected from Xi'an High-tech Zone from March to April 2009, and the air flow rate was 1200 L/min. The PM2.5 were adsorbed to a quartz fiber filter, and after recycled every day, the filter was sonicated with 40 mL of ultrapure water filtered by Milli-Q for 15 min, and repeated 3 times. Thereafter, the suspension was dried in a vacuum freezer and stored at 4° C. Prior to use, PM2.5 was suspended in cell culture medium and sonicated for 30 minutes, and the suspension was filtered with a glass fiber filter to remove debris, to prepare a PM2.5 cell culture solution having a final concentration of 50 μg/mL.

The composition of 12 elements (i.e., S, Ti, Cr, Mn, Fe, Ni, Cu, Zn, As, Br, Mo, Pb) was determined by energy dispersive X-ray fluorescence (ED-XRF). The contents of organic carbon (OC) and elemental carbon (EC) in the samples were analyzed using an organic carbon analyzer (DRI Model 2001 Carbon Analyzer). The results were shown in Table 1.

TABLE 1
Chemical composition of PM2.5 samples collected from Xi'an, China
Elements (unit: μg/m3)

| S | Ti | Cr | Mn | Fe | Ni | Zn | As | Br | Mo | Cd | Pb |
|---|---|---|---|---|---|---|---|---|---|---|---|
| 3.4 | 0.12 | 0.01 | 0.13 | 1.42 | 0 | 2.28 | 0.02 | 0.06 | 0.05 | 0.02 | 0.29 |

Ions (μg/m³)

| K⁺ | Na⁺ | NH₄⁺ | NO₃⁻ | SO₄²⁻ | Mg²⁺ | Ca²⁺ | Cl⁻ | F⁻ |
|---|---|---|---|---|---|---|---|---|
| 1.14 | 1.5 | 4.32 | 9.32 | 13.3 | 0.18 | 2.24 | 3.83 | 0.15 |

Carbon composition (μg/m³)

| Total | | Elemental | Water-soluble organic |
|---|---|---|---|
| carbon | Organic carbon | carbon | carbon |
| 26.7 | 22.43 | 4.27 | 7.62 |

### Analysis of Results:

The composition and source of PM2.5 in different regions are varied. In this experiment, a chemical analysis on PM2.5 samples collected from Xi'an, China was conducted. PM2.5 contains complex components such as metal ions, toxic organic compounds and inorganic substances. The elemental composition analysis showed that S, Zn and Fe were the top three elemental compositions of PM2.5 used in the experiment. Zn and Fe were one of the most abundant crust elements, indicating that dust emission is an important source of the samples. Moreover, high concentrations of S, NO₃⁻ and SO₄²⁻ indicated that coal combustion emissions were another important source. Sulfate and nitrate were mainly from the oxidation of SO₂ and NO_{X} and were believed to be mainly produced by coal combustions in the locality. In addition, studies have shown that the OC/EC of coal combustion emissions is 2.5-10.5, and in this experiment, OC/EC is 5.25, which demonstrates the main contribution of coal combustion emissions. Therefore, we consider the PM2.5 samples to be a typical source of coal and dust.

From the above analysis, we conclude that the composition of the PMs is complex and is affected by the local natural environment. However, in any case, although the PMs have different compositions and contents of harmful substances in different places, but the types of harmful substances are similar, for example, dust is almost an important source of all PMs. Dust is mainly composed of crustal elements, although these elements seem harmless to the human body, if they form PMs, they will be extremely harmful to the human body. One of the hazards of PMs is the damage to the skin barrier.

The hazards of these PMs to human body refer to their effects on the skin, which, on one hand, are manifested in their adverse effects on the skin SC. In some specific embodiments, it is manifested by the direct effect of PMs on the cholesterol metabolism pathway in skin SC, for example, effect of PMs on the elevated cholesterol levels in the SC.

### Example 2: Effect of PM2.5 on In Vitro Cells

2.1 Cell Culture

Primary human epidermal keratinocytes (PC2011, Biocell, Guangdong, China) were cultured in KcGrowth medium (PY1011, Biocell, Guangdong, China), and primary human keratinocytes were cultured in a cell culture incubator at the condition of 37° C. and CO₂ 5%.

The fused keratinocytes were isolated from plates with an EDTA-trypsin solution. After centrifugation, the cells were resuspended in KcGrowth medium at a concentration of 10⁶ cells/ml and then inoculated in a 6-well plate at a density of 2×10⁵/well.

After 24 hours of culture, the medium was removed, and then a PM2.5 suspension with or without green tea extract (GTE) was added to the plate (PM2.5 in Example 1 and 50 μg/mL PM2.5 cell culture medium), three replicates were set for each condition (2 mL of suspension was added to each well and cells were treated with PM2.5 for 24 h). The green tea extract (GTE) had a polyphenol content of 750 μg/ml as measured by the Folin&Ciocalt method using a 20% aqueous solution of 1,3-butanediol having a dry weight of 0.2%. (The GTE stock solution used had a polyphenol content of 750 μg/ml, and a 0.6% GTE stock solution (mass percentage) and PM2.5 suspension (in Example 1) were added to the culture medium. Here, KcGrowth medium was used as a blank control.

2.2 Cell Viability Assay

The fused keratinocytes were separated from the plate with EDTA-trypsin solution in 2.1. After centrifugation, the cells were resuspended in KcGrowth medium at a concentration of 10⁶ cells/ml. The cells were inoculated in a 96-well culture plate at a density of 1×10⁴cells/well and cultured overnight.

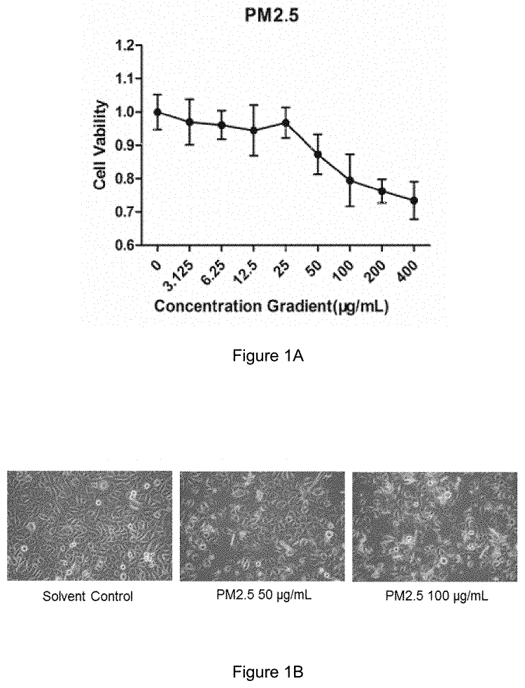

The cells were then treated with different concentrations of PM2.5 for 24 hours (concentration and time shown in FIG. 1). The medium was washed with PBS, and 20 μL of MTT reagent (Formazan) was added to each well, and the plate was incubated in the dark at 37° C. for 4 hours. Finally, the medium was removed and 150 μL of DMSO was added to dissolve the formazan crystals. Absorbance at 490 nm was measured using an ELISA Microplate Reader (BioTeK, USA), to quantify cell viability under different culture conditions.

2.3 Cell Morphology

Cell morphology was observed using an inverted optical microscope (Olympus Corporation, Japan). Keratinocytes cultured in 24-well plates were incubated with PM2.5 or co-treated with GTE solution for 24 hours (FIG. 1). The morphological changes in keratinocytes were observed, with two replicates established under each experimental condition.

### Experimental Results:

A portion of the cultured cells were used in the MTT test to detect changes in morphology or viability. Referring to FIG. 1A and FIG. 1B, experimental results showed that, after cells were cultured with PM2.5 medium at different concentration gradients for 24 hours, the cell viability was observed to decrease significantly with the increase of PM2.5 concentration, when the concentration of PM2 exceeded 50 μg/mL, a large amount of cell fragments appeared (FIG. 1B). Morphologically, after cells were treated with C_{(PM2.5)}>50 μg/mL for 24 hours, the cell viability decreased gradually in a dose-dependent manner (FIG. 1B). When the PM2.5 dose was increased to 50 μg/mL, the cells showed obvious morphological changes, some cell fragments began to appear, cells were shrunk, but the number of adherent cells had no significant change. At a higher concentration of PM2.5, cells were shrunk, rounded and floated, and the number of adherent cells was significantly reduced. Therefore, we chose 50 μg/mL as the maximum safe dose of PM2.5 in the subsequent experiments. At such concentrations, although the cells were damaged, the viability of the cells was not significantly affected, which was advantageous for subsequent experiments. However, in any case, as shown from FIG. 1A, with the gradual increase in the concentration of PM2.5, the viability of cells decreased, and the higher the concentration, the more obvious the rate of decline. This indicated that PM2.5 had a significant effect on cells. Cells are the smallest unit of organisms and important component of SC. The effect on SC is caused by the effect on cells of the organism, which is ultimately manifested as decrease of disappearance of skin barrier functions.

### Example 3: Effect of PM2.5 to mRNA Expression (Transcription Level) in Keratinocytes

After part of keratinocytes were treated by PM2.5 of example 2 (50 μg/mL PM2.5) for 24 hours, at the same time, treated by blank control (without PM2.5) for 24 hours, the total RNA was extracted by TRIzol Reagent (Invitrogen, USA) according to the instructions of manufacturer.

RNA degradation and contamination were tested using a 1% agarose gel and RNA purity was measured using a spectrophotometer (NanoPhotometer, IMPLEN, CA, USA). RNA concentrations were measured using a Qubit RNA Assay Kit in a Qubit 2.0 Flurometer (Life Technologies, CA, USA) and RNA integrity was assessed using the RNA Nano 6000 Assay Kit of Bioanalyzer 2100 System (Agilent Technologies, CA, USA). A cDNA library was prepared using the NEBNext Ultra RNA Library Preparation Kit (NEB, USA) dedicated for Illumina detection. Subsequently, the cDNA concentration was measured by an AMPure XP system (Beckman Coulter, Beverly, USA) to meet the predetermined requirements, and RNA sequencing was performed on Illumina HiSeq 4000 (Illumina, USA) using the Paired-End method.

TABLE 2
RNA concentration and results of integrity test

| | Concentration | | | rRNA Ratio | RNA integrity |
|---|---|---|---|---|---|
| | (ng/μL) | OD260/280 | OD260/230 | [28 s/18 s] | number(RIN) |
| Blank control | 605 | 1.936 | 1.987 | 2.1 | 10(B.02.08) |
| PM2.5 | 588 | 1.96 | 2.162 | 2.1 | 10(B.02.08) |
| GTE + PM2.5 | 578 | 1.986 | 1.571 | 2.5 | 10(B.02.08) |

As shown from Table 2, OD260/280 and OD260/230 were indicative values of nucleic acid purity. The OD260/280 value of pure RNA was 2.0 and OD260/230>2. The results showed that the OD260/280 and OD260/230 values were close to 2.0, indicating no protein or DNA contamination, no contamination of organic solvents, and high RNA purity. This experimental result can be more accurate. OD260/230 and 28S/18S are indicators to measure the integrity of extracted RNAs. If the value of 28s/18s was about 2.0, it indicated that the extracted RNA had good integrity. The results showed that the value of 28s/18s was greater than 2.0, and the 5sRNA band was not visible, indicating that RNA was not degraded. When the RIN value is between 0 and 10, the larger the value, the better the integrity, the result showed that RIN was 10, indicating that RNA had good quality. RNA is the expression of DNA at the transcriptional level, and the purity and integrity of RNA provides reliable guarantee for subsequent experimental results, including the implementation of reverse transcription (cDNA).

HiSeq PE150 was selected as the sequencing strategy and the total sequence obtained was 6G. The ribosomal RNA sequence and the non-coding sequence were removed from the original fragment to obtain a sequence that could be encoded. The gene was determined using HISAT 2.0.4, the quantification of mRNA expression was performed by HTSeq v0.6.1, and the gene differential expression analysis was performed by DEGSeq 1.12.0 and DESeq 1.10.1.

### Results and Analysis:

We performed a comprehensive gene expression profiling of keratinocytes using the Illumina sequencing technology. After rRNA sequences and low expression sequences, were removed, we investigated the changes induced by PM2.5 in human keratinocytes. Compared to the control group (without treatment with PM2.5 and GTE), there was a significant difference in expression in PM2.5 treatment group, defined as Padj (corrected p value)<0.001. As shown from Table 3, compared to the control, 56 genes were significantly up-regulated and 20 genes were significantly down-regulated.

Some significantly up-regulated genes reported in the existing literatures were listed, such as CXCL1, CYP1A1, IL1RN, etc., however, at the same time, the present invention also found increased transcription levels of some undisclosed genes, for example, up-regulation of genes ACLY, ACSS2, HMGCR, HMGCS1, MVD, MSMD1, FDFT1, SQLE, LSS, LDLR, SCD, FASN, INSIGL, etc. (Table 3). These genes were directly related to the cholesterol metabolism pathway, which seemed to indicate that PMs (e.g. PM2.5) were also related to the synthetic route of cholesterol. It would be further analyzed in Example 4.

In addition to genes directly related to cholesterol metabolism, we found for the first time that genes not previously reported to be associated with PM2.5 further included S100A8, S100A9, Krt6b, TXNRD1, FGFBP1, MT2A, CD9, AREG, ITGB1, LAMB3, LAMA3 (see Table 3 for details). Among them, S100A8 and S100A9 belong to the S100 protein family and are distributed in the granular layer, spinous layer and basal layer. Their main function is to involve in the host defense process related to NADPH oxidase activation. Studies have shown that S100A8 and S100A9 played an important role in epidermal wound repair, differentiation and stress response; and they were expressed at very low levels in normal epidermis, but were abundantly expressed in the skin of psoriasis patients and proved to be up-regulated in the skin of patients with atopic dermatitis. Krt6b belongs to the Keratin family and can characterize the division rate of keratinocytes. It is a typical marker of wound healing. Krt6b is up-regulated in skin diseases such as atopic dermatitis and ichthyosis, etc. TXNRD1, encoding thioredoxin reductase, is a typical anti-oxidation gene and is regulated by the Nrf2 transcription factor. FGFBP1 encodes a fibroblast growth factor binding protein that is involved in the regulation of skin cell division and is associated with wound healing and angiogenesis. MT2A is a metal-binding protein that can promote cell proliferation, and it has been found that its expression level is associated with the skin scar formation. CD9 is a cell surface protein that involves in many biological processes, such as wound healing, by coupling the signaling of intracellular signals. AREG amphiregulin plays a role in inflammatory epidermal hyperplasia and sebaceous gland enlargement, and it has been reported that its expression in human airway epithelial cells is up-regulated under the influence of PM2.5. LAM laminin is involved in wound healing and host defense. ITG integrin mediates adhesion of skin cells and is closely related to wound healing and inflammatory response. It is stimulated to maintain skin homeostasis when the skin is subjected to mechanical stress and external damage. We found for the first time that S100A8, S100A9, Krt6b, TXNRD1, FGFBP1, MT2A, CD9, AREG, ITGB1, LAMB3, and LAMA3 were up-regulated in keratinocytes stimulated by PM2.5.

TABLE 3
Genes in keratinocytes that are significantly up-regulated or down-regulated by PM2.5

| | | log2 fold | Significance(PM | |
|---|---|---|---|---|
| Gene | Gene ID | change(PM2.5vsS C) | 2.5vSC) | Description |
| CXCL1 | ENSG00000163739 | 3.3047 | Significant | Chemokine (C-X-C motif) |
| | | | up-regulation | ligand 1 (melanoma growth |
| | | | | stimulating activity, α) |
| CYP1A1 | ENSG00000140465 | 2.114 | Significant | Cytochrome P450 family 1 |
| | | | up-regulation | subfamily 1 |
| S100A8 | ENSG00000143546 | 2.6565 | Significant | S100 calcium-binding protein |
| | | | up-regulation | A8 |
| EIF3CL | ENSG00000205609 | 2.4628 | Significant | Eukaryotic translation |
| | | | up-regulation | initiation factor 3 subunit |
| | | | | C-like |
| S100A9 | ENSG00000163220 | 2.319 | Significant | S100 calcium-binding protein |
| | | | up-regulation | A9 |
| HMGCS1 | ENSG00000112972 | 1.8909 | Significant | 3-hydroxy-3-methylglutaryl |
| | | | up-regulation | coenzyme A synthase 1 |
| IL1RN | ENSG00000136689 | 1.689 | Significant | Interleukin-1 receptor |
| | | | up-regulation | antagonist protein |
| INSIG1 | ENSG00000186480 | 1.5304 | Significant | Insulin-induced gene 1 |
| | | | up-regulation | |
| KRT6B | ENSG00000185479 | 1.3754 | Significant | Keratin 6B, type II |
| | | | up-regulation | |
| TNFAIP3 | ENSG00000118503 | 1.3361 | Significant | TNFα-induced protein 3 |
| | | | up-regulation | |
| SERPINB2 | ENSG00000197632 | 1.451 | Significant | Serine protease inhibitor |
| | | | up-regulation | peptidase inhibitor, type B |
| SOD2 | ENSG00000112096 | 1.3093 | Significant | Superoxide dismutase 2 |
| | | | up-regulation | |
| NFKBIA | ENSG00000100906 | 1.2489 | Significant | Nuclear factor of κ light |
| | | | up-regulation | chain polypeptide gene |
| | | | | enhancer in B cell inhibitory |
| | | | | factor α |
| TXNRD1 | ENSG00000198431 | 1.2116 | Significant | Thioredoxin reductase 1 |
| | | | up-regulation | |
| TACSTD2 | ENSG00000184292 | 1.1573 | Significant | Tumor-associated calcium |
| | | | up-regulation | signaling 2 |
| HBEGF | ENSG00000113070 | 1.1016 | Significant | Heparin-binding EGF-like |
| | | | up-regulation | growth factor |
| MSM01 | ENSG00000052802 | 1.0632 | Significant | Methyl sterol |
| | | | up-regulation | monooxygenase 1 |
| FASN | ENSG00000169710 | 1.0064 | Significant | Fattyacid synthetase |
| | | | up-regulation | |
| ACFY | ENSG00000131473 | 0.99489 | Significant | ATP citrate lyase |
| | | | up-regulation | |
| AFDH1A3 | ENSG00000184254 | 0.98715 | Significant | Aldehyde dehydrogenase |
| | | | up-regulation | family member |
| SQFE | ENSG00000104549 | 0.9585 | Significant | Squalene epoxidase |
| | | | up-regulation | |
| ACSS2 | ENSG00000131069 | 0.95247 | Significant | Acyl-CoA synthetase |
| | | | up-regulation | short chain |
| FGFBP1 | ENSG00000137440 | 0.95138 | Significant | Fibroblast growth factor |
| | | | up-regulation | binding protein 1 |
| FTL | ENSG00000087086 | 0.93974 | Significant | Ferritin, light chain |
| | | | up-regulation | polypeptide |
| BHEHE40 | ENSG00000134107 | 0.89669 | Significant | Basic Spiral - Ring - |
| | | | up-regulation | Spiral Family 40 |
| KRT19 | ENSG00000171345 | 0.87714 | Significant | Keratin 19 |
| | | | up-regulation | |
| HMGCR | ENSG00000113161 | 0.8467 | Significant | 3-hydroxy-3-methylgluta |
| | | | up-regulation | ryl-COA reductase |
| IL1B | ENSG00000125538 | 0.83678 | Significant | Interleukin-1 β |
| | | | up-regulation | |
| TFPI2 | ENSG00000105825 | 0.8355 | Significant | Tissue factor pathway |
| | | | up-regulation | inhibitor 2 |
| INHBA | ENSG00000122641 | 0.82203 | Significant | Inhibin βA |
| | | | up-regulation | |
| MT2A | ENSG00000125148 | 0.78451 | Significant | Metallothionein 2A |
| | | | up-regulation | |
| MVD | ENSG00000167508 | 0.75521 | Significant | Mevalonic acid |
| | | | up-regulation | diphosphate decarboxylase |
| ACTG1 | ENSG00000184009 | 0.71558 | Significant | Actinγl |
| | | | up-regulation | |
| LDLR | ENSG00000130164 | 0.69696 | Significant | LDL receptor |
| | | | up-regulation | |
| LSS | ENSG00000160285 | 0.68795 | Significant | lanosterol |
| | | | up-regulation | synthase(2,3-oxidized |
| | | | | squalene - lanosterol cyclase) |
| SCD | ENSG00000099194 | 0.59881 | Significant | Stearoyl-CoA desaturase |
| | | | up-regulation | (Δ-9-desaturase) |
| FDFT1 | ENSG00000079459 | 0.59669 | Significant | Faractone-diphosphate |
| | | | up-regulation | lactone transferase 1 |
| CD9 | ENSG00000010278 | 0.54031 | Significant | CD9 molecule |
| | | | up-regulation | |
| TUBB6 | ENSG00000176014 | 0.52809 | Significant | Tubulinβ 6 V |
| | | | up-regulation | |
| TINAGL1 | ENSG00000142910 | 0.51832 | Significant | Tubular interstitial |
| | | | up-regulation | nephritis antigen-like type 1 |
| FERMT1 | ENSG00000101311 | 0.49405 | Significant | Ferritin family member 1 |
| | | | up-regulation | |
| SERPINB5 | ENSG00000206075 | 0.4714 | Significant | Serine protein inhibitor, |
| | | | up-regulation | branch B |
| S100A2 | ENSG00000196754 | 0.45788 | Significant | S100 calcium-binding |
| | | | up-regulation | protein A2 |
| ILIA | ENSG00000115008 | 0.42438 | Significant | Interleukin 1A |
| | | | up-regulation | |
| S100A6 | ENSG00000197956 | 0.42428 | Significant | S100 calcium-binding |
| | | | up-regulation | protein A6 |
| TMSB10 | ENSG00000034510 | 0.36556 | Significant | Thymosinβ10 |
| | | | up-regulation | |
| KRT6A | ENSG00000205420 | 0.32895 | Significant | Keratin 6A, type II |
| | | | up-regulation | |
| LAMC2 | ENSG00000058085 | 0.32448 | Significant | Laminin subunit γ2 |
| | | | up-regulation | |
| AREG | ENSG00000109321 | 0.31693 | Significant | Amphiregulin |
| | | | up-regulation | |
| SFN | ENSG00000175793 | 0.31607 | Significant | Human stratifin |
| | | | up-regulation | |
| ITGB1 | ENSG00000150093 | 0.30003 | Significant | Integrin subunitβ1 |
| | | | up-regulation | |
| ACTB | ENSG00000075624 | 0.26656 | Significant | Actinβ |
| | | | up-regulation | |
| LAMB3 | ENSG00000196878 | 0.17649 | Significant | Laminin subunit β3 |
| | | | up-regulation | |
| LAMA3 | ENSG00000053747 | 0.16937 | Significant | Laminin subunit α3 |
| | | | up-regulation | |
| KRT5 | ENSG00000186081 | 0.11129 | Significant | Keratin 5, type II |
| | | | up-regulation | |
| PLEC | ENSG00000178209 | −0.12597 | Significant | Reticulin |
| | | | down-regulation | |
| UPP1 | ENSG00000183696 | −0.27439 | Significant | Uridine phosphorylase 1 |
| | | | down-regulation | |
| ITGB4 | ENSG00000132470 | −0.27773 | Significant | Integrin subunitβ4 |
| | | | down-regulation | |
| H3F3B | ENSG00000132475 | −0.33111 | Significant | H3 histone, 3B family |
| | | | down-regulation | |
| TP63 | ENSG00000073282 | −0.39204 | Significant | Tumor protein p63 |
| | | | down-regulation | |
| ARF6 | ENSG00000165527 | −0.39607 | Significant | ADPribosylationfactor6 |
| | | | down-regulation | |
| AJUBA | ENSG00000129474 | −0.39871 | Significant | ajubaLIMprotein |
| | | | down-regulation | |
| TRIB3 | ENSG00000101255 | −0.41682 | Significant | tribbles pseudokinase3 |
| | | | down-regulation | |
| MYC | ENSG00000136997 | −0.42793 | Significant | v-mycavian cell tumor |
| | | | down-regulation | virus oncogene homologue |
| GAS5 | ENSG00000234741 | −0.48023 | Significant | Growth arrest |
| | | | down-regulation | specificity5 |
| FN1 | ENSG00000115414 | −0.52053 | Significant | Fibronectin1 |
| | | | down-regulation | |
| THBS1 | ENSG00000137801 | −0.5431 | Significant | Thrombospondin1 |
| | | | down-regulation | |
| SERPINE2 | ENSG00000135919 | −0.6276 | Significant | Serine protease inhibitor |
| | | | down-regulation | peptidase inhibitor, clade E |
| C6orf48 | ENSG00000204387 | −0.67374 | Significant | Chromosome 6 open |
| | | | down-regulation | reading frame 48 |
| ZFAS1 | ENSG00000177410 | −0.69429 | Significant | ZNFXlantisenseRNA 1 |
| | | | down-regulation | |
| THBS2 | ENSG00000186340 | −0.75684 | Significant | Thrombospondin 2 |
| | | | down-regulation | |
| SNHG1 | ENSG00000255717 | −0.77884 | Significant | Small nucleolar RNA |
| | | | down-regulation | host gene 1 |
| SNHG5 | ENSG00000203875 | −0.89836 | Significant | Small nucleolar RNA |
| | | | down-regulation | host gene 5 |
| H1F0 | ENSG00000189060 | −0.94606 | Significant | H1histone family |
| | | | down-regulation | member 0 |
| TXNIP | ENSG00000265972 | −1.0801 | Significant | Thioredoxin interacting |
| | | | down-regulation | protein |

### Example 4: Functional Annotation and Cellular Pathway Analysis of Keratinocytes Treated with PM2.5

Metascape (http://metascape.org/gp/index.html#/main/step1) is used for bioinformatics analysis to summarize and visualize raw data at the level of gene transcription. To gain a deep understanding of the unique pathways and protein networks that respond to PM2.5 stimuli, we used the online database Metascape to analyze gene ontology (GO) molecular entries (biological processes, molecular function and cellular localization) and KEGG pathways of significantly up-regulated and down-regulated genes (http://metascape.org/gp/index.html#/main/step1).

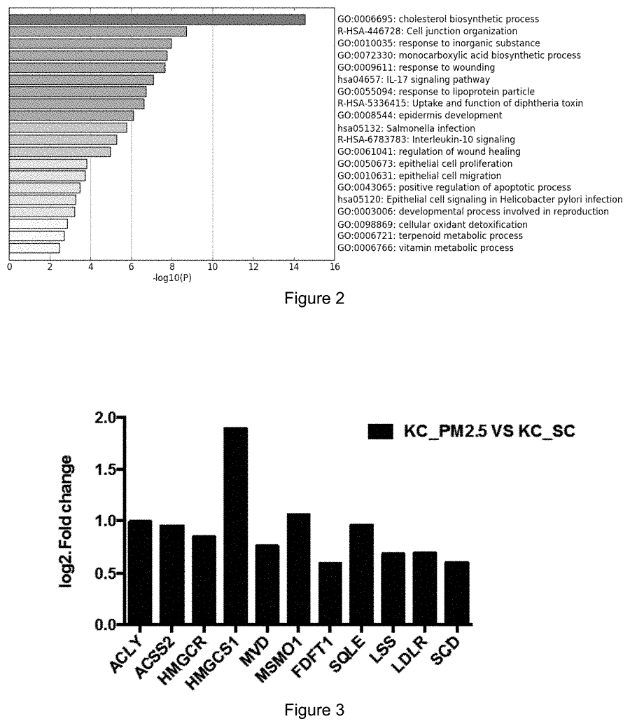

Through bioinformatics analysis, the information such as biochemical pathways involved in changing genes, subcellular localization of end products of gene expressions and correlation with some diseases can be obtained. The genes that were significantly up-regulated compared to the control group after PM2.5 treatment (pad j<0.001) were used for analysis. The results were shown in FIG. 2, among the top 20 GO entries and routes, the most significantly enriched GO entry was the cholesterol biosynthesis process (GO:GO: 0006695), indicating that PM2.5 was highly likely to affect cholesterol biosynthesis or PM2.5 was directly related to the metabolism of cholesterol. In addition, the response to lipoprotein particles (GO:0055094) was also one of the most enriched entries, indicating there existed a close relationship between PM2.5 and cholesterol in cells. Further, inflammation-related pathways such as the IL-17 signaling pathway (hsa04657) and interleukin-10 signaling (R-HSA-6783783) were also highly enriched. At the same time, the response to wound healing (GO: 0009611) and the regulation of wound healing (GO: 0061041) also ranked the top 20, indicating the stimulation of skin inflammatory responses. The positive regulation of the apoptotic process (GO:0043065) and the cellular oxidant detoxification (GO:0098869) pathway revealed that PM2.5 could induce apoptosis and intracellular oxidative clearance as defense responses.

In short, through functional annotation analysis, the cholesterol biosynthesis pathway, inflammatory and oxidative stress pathway, and closely related apoptotic pathway are the most significantly stimulated pathways in keratinocytes treated with PM2.5 and are also directly associated with PM2.5. (FIG. 2). GO analysis showed abnormal cholesterol metabolism in keratinocytes after treated with PM2.5. Because lipids are a major component of the cell membrane and an important component of SC, it is essential to maintain normal functions of the skin barrier. This suggests that PM2.5 changes the cholesterol metabolism pathway, which causes normal metabolism of cholesterol and changes in SC, thereby impairing the barrier functions of the skin.

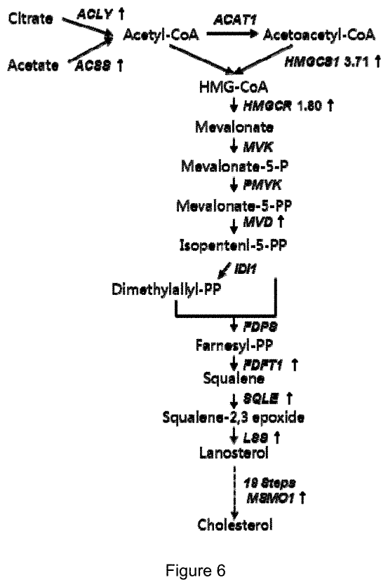

According to the transcriptomics results, the “cholesterol biosynthesis process” and “response to lipoprotein particles” ranked the most significantly enriched entries in the GO analysis, we further analyzed the variant genes involved in the entries. Some of the genes associated with cholesterol metabolism were listed in Table 4, and the changes in expression levels of the genes compared to the controls were plotted in FIG. 3. The cholesterol biosynthesis pathway was shown in FIG. 6. According to FIG. 3, Table 4 and FIG. 6, PM2.5 could significantly change the content of direct substances in the cholesterol metabolism route, and also indirectly affect the changes of main enzymes in the cholesterol metabolism route, causing changes in the entire route and eventually up-regulating the cholesterol level (relative to untreated normal SC cells (controls)). This further provided that PM2.5 has a direct correlation with the cholesterol metabolism in SC, which is a new discovery of the present invention.

In general, for example, as shown in FIG. 6, the cholesterol metabolism process is divided into three stages. Firstly, acetyl-CoA forms mevalonic acid; secondly, two mevalonic acids are condensed into isoprene and to form squalene; and thirdly, squalene is converted to cholesterol. Both cholesterol and fatty acid synthesis begin with the common precursor acetyl-CoA, which is derived from the catabolism of carbohydrates, proteins and lipids.

Among the genes induced by PM2.5, 13 genes were significantly up-regulated, as shown in Table 4, FIG. 3 and FIG. 6. ACLY and ACSS2 were involved in the production of acetyl-CoA. HMGCS1 and HMGCR were involved in the first stage of cholesterol metabolism. MVD and FDFT1 were involved in the second stage of cholesterol metabolism. LSS and SQLE were involved in the synthesis of lanosterol from squalene in the third stage of cholesterol metabolism. LDLR encodes a protein receptor that binds to the carrier of cholesterol-LDL, which is essential for the uptake of cholesterol into cells. Therefore, the up-regulation of these genes under stimulation of PM2.5 may lead to an increase in cholesterol metabolism. As shown from FIG. 3, 13 genes were significantly up-regulated, which affected the synthesis route of cholesterol and finally increased the cholesterol level. In addition, INSIG1 plays an important role in regulating cholesterol metabolism in the skin, by regulating the transcription of the transcription factor SREBP/SCAP (a regulatory factor of cholesterol and fatty acid synthesis) and HMG-CoA reductase (rate-limiting enzyme of cholesterol metabolism), the steady state of cholesterol in the skin was maintained. FASN, as another major rate-limiting enzyme of barrier lipid fatty acid synthesis, has also been shown to be closely related to cholesterol synthesis. The up-regulation of FASN activity can promote cholesterol production by regulating acetyl-CoA. This fully demonstrates that PM2.5 can directly or indirectly change the gene expression or the activity or quantity of enzymes, or the number of substrates regardless of the levels of genes, enzymes or substrates, there by ultimately increasing the cholesterol level relative to the controls. Thus, it proves that the damage of skin barrier is directly related to PM2.5. In addition, if some external active ingredients can be reversed, for example, down-regulating one or more of up-regulated genes, or lowering the activity of the enzymes, or decreasing or increasing the precursor or substrate, these changes will eventually cause the cholesterol levels to be at a normal state. These active ingredients are effective substances.

TABLE 4
Cholesterol metabolism-related genes and corresponding GO entries

| | | | Corrected | | | |
|---|---|---|---|---|---|---|
| | Log | | P value | | Biological Process | |
| Gene | 2(difference fold) | P value | (Padjue) | Description | (GO) | Category |
| ACLY | 0.99489 | 6.19E−24 | 1.04E−20 | ATP citrate lyase | GO: 0035338 | Acetyl-coen |
| | | | | | Long chain fatty | zyme |
| | | | | | acyl- CoA | synthesis |
| | | | | | biosynthesis | |
| | | | | | process: | |
| | | | | | GO: 0046949: | |
| | | | | | Fatty acyl- CoA | |
| | | | | | biosynthesis | |
| | | | | | process: | |
| | | | | | GO: 0035336 | |
| | | | | | Fatty acyl- CoA | |
| | | | | | metabolic process | |
| ACSS2 | 0.95247 | 2.72E−10 | 1.75E−07 | Acyl -CoA | GO: 0019542 | |
| | | | | synthetase short | propionic acid | |
| | | | | chain family | biosynthesis | |
| | | | | member 2 | process: | |
| | | | | | GO : 0019427 | |
| | | | | | Acetyl acyl- CoA | |
| | | | | | biosynthesis | |
| | | | | | process: | |
| | | | | | GO: 0051790 | |
| | | | | | short-chain fatty | |
| | | | | | acid biosynthesis | |
| | | | | | process | |
| HMGCR | 0.8467 | 2.49E−08 | 1.13E−05 | 3-hydroxy- | GO: 0061179 | Cholesterol |
| | | | | 3-methylglutaryl- | Negative | synthesis |
| | | | | COA reductase | regulation of | step 1 |
| | | | | | insulin secretion | |
| | | | | | and involve in the | |
| | | | | | response of cells | |
| | | | | | to glucose | |
| | | | | | stimulation; | |
| | | | | | GO: 00 10666: | |
| | | | | | Positive regulation | |
| | | | | | of cardiomyocyte | |
| | | | | | apoptosis; | |
| | | | | | GO: 0046676 | |
| | | | | | Negative | |
| | | | | | regulation of | |
| | | | | | insulin secretion | |
| HMGCSI | 1.8909 | 4.51E−42 | 1.64E−38 | 3 -hy droxy -3 -methy | GO: 00 46690: | |
| | | | | lglutaryl- COA | Reaction to | |
| | | | | synthase 1 | strontium ions; | |
| | | | | | GO: 0009645 | |
| | | | | | Reaction to low | |
| | | | | | light intensity | |
| | | | | | stimuli; | |
| | | | | | GO: 0071397 | |
| | | | | | Cellular response | |
| | | | | | to cholesterol | |
| MVD | 0.75521 | 6.24E−07 | 2.22E−04 | Mevalonate | GO: 0006489 | Cholesterol |
| | | | | disulfate | diethyl | synthesis |
| | | | | decarboxylase | diphosphate | step 2 |
| | | | | | biosynthesis | |
| | | | | | process; | |
| | | | | | GO: 0019287 | |
| | | | | | isoprene | |
| | | | | | diphosphate | |
| | | | | | biosynthesis | |
| | | | | | process, | |
| | | | | | mevalonate route; | |
| | | | | | GO: 0046465 | |
| | | | | | dipropyl | |
| | | | | | diphosphate | |
| | | | | | metabolic process | |
| MSMO1 | 1.0632 | 3.23E−20 | 5.04E−17 | Methyl sterol | GO: 0006695 | |
| | | | | monooxygenase 1 | cholesterol | |
| | | | | | biosynthesis | |
| | | | | | process; | |
| | | | | | GO: 1902653 | |
| | | | | | secondary alcohol | |
| | | | | | biosynthesis | |
| | | | | | process; | |
| | | | | | GO: 0016126 | |
| | | | | | sterol biosynthesis | |
| | | | | | process | |
| FDFT1 | 0.59669 | 4.65E−12 | 3.51E−09 | Jaceosidin- | GO: 0045338 | |
| | | | | jaceosidin disulfate | farnesyl | |
| | | | | transferase 1 | diphosphate | |
| | | | | | metabolic process; | |
| | | | | | GO: 0006696 | |
| | | | | | ergosterol | |
| | | | | | biosynthesis | |
| | | | | | process; | |
| | | | | | GO: 0008204 | |
| | | | | | sterol biosynthesis | |
| | | | | | process; | |
| SQLE | 0.9585 | 6.93E−11 | 4.74E−08 | Squalene epoxidase | GO: 0006695 | Cholesterol |
| | | | | | cholesterol | synthesis |
| | | | | | biosynthesis | step 3 |
| | | | | | process; | |
| | | | | | GO: 1902653 | |
| | | | | | secondary alcohol | |
| | | | | | biosynthesis | |
| | | | | | process; | |
| | | | | | GO: 0016126 | |
| | | | | | sterol biosynthesis | |
| | | | | | process | |
| LSS | 0.68795 | 3.73E−08 | 1.66E−05 | Lanosterol | GO: 0006695 | |
| | | | | synthase | cholesterol | |
| | | | | | biosynthesis | |
| | | | | | process; | |
| | | | | | GO: 1902653 | |
| | | | | | secondary alcohol | |
| | | | | | biosynthesis | |
| | | | | | process; | |
| | | | | | GO: 0016126 | |
| | | | | | sterol biosynthesis | |
| | | | | | process | |
| LDLR | 0.69696 | 2.72E−12 | 2.20E−09 | Low density | GO: 0010899 | Cholesterol |
| | | | | lipoprotein receptor | adjust the | transport |
| | | | | | catabolic process | |
| | | | | | of | |
| | | | | | phosphatidylcholine; | |
| | | | | | GO: 0090118 | |
| | | | | | Receptor-mediated | |
| | | | | | endocytosis is | |
| | | | | | involved in | |
| | | | | | cholesterol | |
| | | | | | transport; | |
| | | | | | GO: 0010867 | |
| | | | | | Positive regulation | |
| | | | | | of triglyceride | |
| | | | | | biosynthesis | |
| | | | | | process | |
| SCD | 0.59881 | 7.25E−61 | 5.29E−57 | Stearoyl-CoA | GO: 0035338 long | Fatty acid |
| | | | | desaturase | chain fatty acyl- | synthesis |
| | | | | | CoA biosynthesis | |
| | | | | | process; | |
| | | | | | GO: 0046949 fatty | |
| | | | | | acyl- CoA | |
| | | | | | biosynthesis | |
| | | | | | process; | |
| | | | | | GO: 0035336 long | |
| | | | | | chain fatty acyl- | |
| | | | | | CoA metabolic | |
| | | | | | process | |
| FASN | 1.0064 | 1.30E−89 | 2.83E−85 | Fatty acid synthase | GO: 0035338 long | |
| | | | | | chain fatty acyl- | |
| | | | | | CoA biosynthesis | |
| | | | | | process; | |
| | | | | | GO: 0046949 fatty | |
| | | | | | acyl- CoA | |
| | | | | | biosynthesis | |
| | | | | | process; | |
| | | | | | GO: 0015939 | |
| | | | | | Pantothenic acid | |
| | | | | | metabolism | |
| | | | | | process | |
| INSIG1 | 1.5304 | 9.01E−11 | 5.98E−08 | Insulin-induced | GO: 19011303 | |
| | | | | gene 1 | Load-regulated | |
| | | | | | cargo loaded into | |
| | | | | | COPII coated | |
| | | | | | vesicles; | |
| | | | | | GO: 1901301 | |
| | | | | | regulates the | |
| | | | | | loading of cargo | |
| | | | | | into COPII coated | |
| | | | | | vesicles; | |
| | | | | | GO: 0045717 | |
| | | | | | Negative | |
| | | | | | regulation of fatty | |
| | | | | | acid biosynthesis | |

### Example 5: Effect of Tea Polyphenols on mRNA Expressions (Transcription Level) in Keratinocytes Stimulated by PM2.5

Keratinocytes were treated with GTE (0.6%)+PM2.5 (50 μg/mL) for 24 hours, then RNAs were extracted and sequenced according to the method in Example 3.

When cells were treated with PM2.5 and GTE (tea polyphenols) simultaneously, it was found that the effect of PM2.5 on keratinocyte transcription levels was effectively reversed. The specific results were shown in Table 5. Compared with cells treated with PM2.5 alone, the addition of GTE resulted in the significant up-regulation of 22 genes and the significant down-regulation of 52 genes. The changes of these genes involved the changes in multiple metabolic processes.

Tea polyphenols affect a number of pathways closely related to inflammatory responses. Among the significantly down-regulated genes involved, IL-la is a recognized inflammatory marker and is closely related to the occurrence of acne; matrix metalloproteinases-1 (MMP-1), as a biomarker for collagen degradation, is induced under thermal conditions or UV stress; for numerous HSP families, including heat shock protein 90 alpha family class A member 1 (HSP9OAA1), heat shock protein family A (HSP70) member 8 (HSPA8), their expressions will be stimulated under different stresses or external stimuli to protect the skin; S100A9 (calgranulin B or MRP-14) is a model molecule closely related to damage, which is up-regulated in many inflammatory skin diseases. Therefore, tea polyphenols mainly relieve the inflammatory responses induced by PM2.5.

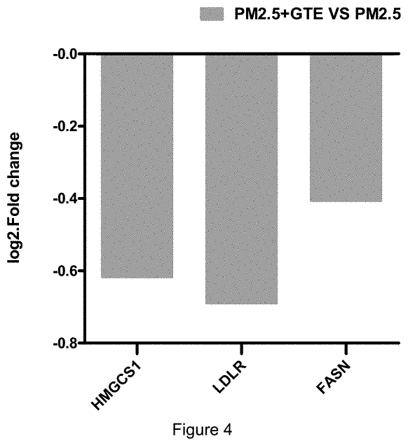

The regulatory effect of GTE on the above genes has been reported in the existing literatures. However, we found for the first time that the addition of GTE (tea polyphenols) to the PM2.5-treated cells would significantly down-regulate the expressions of HMGCS1, LDLR and FASN in the cholesterol metabolism pathway, as shown in FIG. 4. These genes were significantly up-regulated when treated with PM2.5 alone (as shown in Table 3). This suggested that the protective effect of GTE (tea polyphenols) on cells was to reverse the adverse effects of PM2.5 on cholesterol metabolism of keratinocytes, thereby maintaining cholesterol metabolism in a stable state. This further indicated that tea polyphenols could reverse the effect of PMs on the synthesis of cholesterol in skin SC, which would be further demonstrated in detail later. At the same time, we conducted similar experiments using other sources of polyphenols (plant extracts, microbial fermentation, animal sources, of which plant sources include Camellia sinensis, apples, etc.) and found that it could reverse the expression levels of some genes under the PM2.5 stimulation conditions shown in Table 3, which mainly down-regulated the expressions of HMGCS1, LDLR and FASN (experimental data were omitted). This further confirmed that, any substance that can down-regulate the expression levels of genes in FIG. 3 can reverse the adverse effects of PM2.5 on the skin and help maintain cholesterol at normal levels. It is also demonstrated from another aspect that if the substance related to the cholesterol anabolic pathway changes, it indicates that the changes are caused by the adverse effects of PMs on the skin, providing a new evidence of adverse effect of PMs on the skin. Because there are numerous substances affecting the cholesterol metabolism pathway and the influencing factors are extremely complicated, at least we can take the effect of PM2.5 as a possibility when analyzing the cholesterol metabolism of SC. Thus, it can be specifically used to develop or screen some active ingredients that have an adverse effect on the skin due to PM2.5. These active ingredients are used to improve and repair the skin as a new way of skin damage caused by PMs, for example, PM2.5.

TABLE 5
Significantly up-regulated and down-regulated genes after treatment
of keratinocytes with GTE + PM2.5 vs. PM2.5 alone.

| | | Log2 fold | | |
|---|---|---|---|---|
| | | change(PM2.5+GTE | Significance(PM2.5 + GT | |
| Gene | Gene ID | vsPM2.5) | EvsPM2.5) | Description |
| TGFBI | ENSG00000120708 | 1.4492 | Significant | Transforming |
| | | | up-regulation | growth factor, β |
| | | | | induced |
| CYP1A1 | ENSG00000140465 | 1.234 | Significant | Cytochrome |
| | | | up-regulation | P450 family 1 |
| | | | | subfamily |
| | | | | memberl |
| PMEPA1 | ENSG00000124225 | 0.99561 | Significant | Prostate |
| | | | up-regulation | transmembrane |
| | | | | protein, androgen- |
| | | | | induced 1 |
| FLRT2 | ENSG00000185070 | 0.97781 | Significant | Fibronectin |
| | | | up-regulation | leucine-rich |
| | | | | transmembrane |
| | | | | protein 2 |
| ITGB6 | ENSG00000115221 | 0.93237 | Significant | Integrin |
| | | | up-regulation | subunit β6 |
| COL7A1 | ENSG00000114270 | 0.92873 | Significant | Collagen, type |
| | | | up-regulation | VII, al |
| TSC22 | ENSG00000157514 | 0.77568 | Significant | TSC22domain |
| D3 | | | up-regulation | name family |
| | | | | member3 |
| SERPINE1 | ENSG00000106366 | 0.74767 | Significant | Serine |
| | | | up-regulation | protease inhibitor |
| | | | | peptidase |
| | | | | inhibitor, clade E |
| FN1 | ENSG00000115414 | 0.60798 | Significant | Fibronectin 1 |
| | | | up-regulation | |
| TXNIP | ENSG00000265972 | 0.55952 | Significant | Thioredoxin |
| | | | up-regulation | interacting protein |
| HSPB1 | ENSG00000106211 | 0.5207 | Significant | HSP family B |
| | | | up-regulation | (small protein) |
| | | | | member 1 |
| KRT16 | ENSG00000186832 | 0.51764 | Significant | Keratin 16, |
| | | | up-regulation | type I |
| FAT2 | ENSG00000086570 | 0.41143 | Significant | FAT atypical |
| | | | up-regulation | cadherin 2 |
| NEAT1 | ENSG00000245532 | 0.3657 | Significant | Para-nuclear |
| | | | up-regulation | axis assembly |
| | | | | transcript 1 |
| KRT14 | ENSG00000186847 | 0.26988 | Significant | Keratin 14, |
| | | | up-regulation | type I |
| KRT17 | ENSG00000128422 | 0.22845 | Significant | Keratin 17, |
| | | | up-regulation | type I |
| DSP | ENSG00000096696 | 0.20788 | Significant | Desmosome |
| | | | up-regulation | |
| SLC7A5 | ENSG00000103257 | 0.20528 | Significant | Solute carrier |
| | | | up-regulation | family 7 |
| THBS1 | ENSG00000137801 | 0.15224 | Significant | Thrombospon |
| | | | up-regulation | din 1 |
| KRT5 | ENSG00000186081 | 0.14861 | Significant | Keratin5, type |
| | | | up-regulation | II |
| LAMA3 | ENSG00000053747 | 0.11887 | Significant | Laminin |
| | | | up-regulation | subunit a3 |
| LAMC2 | ENSG00000058085 | 0.067516 | Significant | Laminin |
| | | | up-regulation | subunit γ2 |
| PLEC | ENSG00000178209 | −0.19829 | Significant | Reticulin |
| | | | down-regulation | |
| ACTB | ENSG00000075624 | −0.24413 | Significant | Actin, β |
| | | | down-regulation | source |
| ANXA2 | ENSG00000182718 | −0.26389 | Significant | AnnexinA2 |
| | | | down-regulation | |
| FLNB | ENSG00000136068 | −0.28498 | Significant | FilamentinB |
| | | | down-regulation | |
| FSCN1 | ENSG00000075618 | −0.34044 | Significant | Actin - actin - |
| | | | down-regulation | protein 1 |
| HMGA1 | ENSG00000137309 | −0.40461 | Significant | High mobility |
| | | | down-regulation | group AT-h00k 1 |
| FASN | ENSG00000169710 | −0.40569 | Significant | Fatty acid |
| | | | down-regulation | synthetase |
| F3 | ENSG00000117525 | −0.41832 | Significant | Coagulation factor |
| | | | down-regulation | III |
| SDC1 | ENSG00000115884 | −0.45119 | Significant | Syndecan |
| | | | down-regulation | 1 (heparan sulfate |
| | | | | protein encoding |
| | | | | gene) |
| UPP1 | ENSG00000183696 | −0.45218 | Significant | Uridine |
| | | | down-regulation | phosphorylasel |
| ACTG1 | ENSG00000184009 | −0.48989 | Significant | Actin γ1 |
| | | | down-regulation | |
| HSP90AA1 | ENSG00000080824 | −0.52262 | Significant | HSP90kDa a |
| | | | down-regulation | family member1 |
| DCBLD2 | ENSG00000057019 | −0.52295 | Significant | Discoid protein, |
| | | | down-regulation | Disc-based |
| | | | | protein, CUB and |
| | | | | LCCL domains |
| | | | | containing 2 |
| MCL1 | ENSG00000143384 | −0.52793 | Significant | Myeloid cell |
| | | | down-regulation | leukemia 1 |
| AREG | ENSG00000109321 | −0.53463 | Significant | Amphiregulin |
| | | | down-regulation | |
| PLAU | ENSG00000122861 | −0.56674 | Significant | Plasminogen |
| | | | down-regulation | activator, |
| | | | | urokinase |
| VEGFA | ENSG00000112715 | −0.58028 | Significant | Vascular |
| | | | down-regulation | endothelial growth |
| | | | | factor A. |
| PHLDA1 | ENSG00000139289 | −0.59647 | Significant | Pleckstrin |
| | | | down-regulation | (platelet-associate |
| | | | | d protein) |
| | | | | homology domain, |
| | | | | family A, member |
| | | | | 1 |
| G0S2 | ENSG00000123689 | −0.60398 | Significant | GO / G1 regulatory |
| | | | down-regulation | protein 2 |
| HMGCSI | ENSG00000112972 | −0.61682 | Significant | Hydroxymethylglu |
| | | | down-regulation | taryl coenzyme A |
| | | | | synthase |
| ILIA | ENSG00000115008 | −0.62145 | Significant | Interleukinl a(IL-1 |
| | | | down-regulation | a) |
| CDCP1 | ENSG00000163814 | −0.62979 | Significant | UBdomain |
| | | | down-regulation | containing protein |
| | | | | 1 |
| HSPA8 | ENSG00000109971 | −0.64266 | Significant | HSP family A |
| | | | down-regulation | (Hsp70) member 8 |
| F0SL1 | ENSG00000175592 | −0.67408 | Significant | FOS-like antigen 1 |
| | | | down-regulation | |
| IFITM3 | ENSG00000142089 | −0.68712 | Significant | Interferon-induced |
| | | | down-regulation | transmembrane |
| | | | | protein 3 |
| LDLR | ENSG00000130164 | −0.68895 | Significant | LDL receptor |
| | | | down-regulation | |
| SEMA4B | ENSG00000185033 | −0.70395 | Significant | Serna domain, |
| | | | down-regulation | immunoglobulin |
| | | | | domain (Ig), |
| | | | | transmembrane |
| | | | | domain (TM) and |
| | | | | short cytoplasmic |
| | | | | domain |
| | | | | (semaphorin) 4B |
| FGFBP1 | ENSG00000137440 | −0.75673 | Significant | Fibroblast growth |
| | | | down-regulation | factor binding |
| | | | | protein 1 |
| EHD1 | ENSG00000110047 | −0.78284 | Significant | EH domain |
| | | | down-regulation | containing 1 |
| AQP3 | ENSG00000165272 | −0.79588 | Significant | Aquaporin 3 |
| | | | down-regulation | |
| TNFAIP3 | ENSG00000118503 | −0.80529 | Significant | TNFa-induced |
| | | | down-regulation | protein 3 |
| LY6E | ENSG00000160932 | −0.84867 | Significant | Lymphocyte |
| | | | down-regulation | antigen 6 |
| | | | | complex, locus E |
| RN7SL1 | ENSG00000283029 | −0.85558 | Significant | RNA, 7SL |
| | | | down-regulation | |
| ANTXR2 | ENSG00000163297 | −0.87628 | Significant | Anthrax toxin |
| | | | down-regulation | receptor2 |
| 0AS3 | ENSG00000111331 | −0.88431 | Significant | 2′-5′-oligoadenylate |
| | | | down-regulation | synthetase 3 |
| IFI27 | ENSG00000165949 | −0.91758 | Significant | Interferon, |
| | | | down-regulation | α-induced |
| | | | | protein 27 |
| MT1E | ENSG00000169715 | −E0018 | Significant | Metallothionein |
| | | | down-regulation | |
| TGFA | ENSG00000163235 | −E003 | Significant | Transforming |
| | | | down-regulation | growth factor a |
| ERRFI1 | ENSG00000116285 | −E0035 | Significant | ERBB receptor |
| | | | down-regulation | feedback |
| | | | | inhibitor 1 |
| ADAM8 | ENSG00000151651 | −E0092 | Significant | ADAM metal |
| | | | down-regulation | peptidase domain 8 |
| TFPI2 | ENSG00000105825 | −1.0404 | Significant | Tissue factor |
| | | | down-regulation | pathway inhibitor2 |
| IFI6 | ENSG00000126709 | −1.0827 | Significant | Interferon, |
| | | | down-regulation | α-induced protein 6 |
| EREG | ENSG00000124882 | −1.0885 | Significant | Epiregulin |
| | | | down-regulation | |
| OAS2 | ENSG00000111335 | −1.1442 | Significant | 2′-5′-oligoadenylat |
| | | | down-regulation | e synthetase 2 |
| HBEGF | ENSG00000113070 | −1.272 | Significant | Heparin-binding |
| | | | down-regulation | EGF-like growth |
| | | | | factor |
| | | | | [Source:HGNC |
| | | | | Symbol;Acc:HGN |
| | | | | C:3059] |
| CITED4 | ENSG00000179862 | −1.2741 | Significant | Cbp / |
| | | | down-regulation | p300-interacting |
| | | | | transactivator with |
| | | | | a carboxy-terminal |
| | | | | domain rich in Glu/ |
| | | | | Asp, 4 |
| MT2A | ENSG00000125148 | −1.2851 | Significant | Metallothionein2A |
| | | | down-regulation | |
| FAM110C | ENSG00000184731 | −1.3032 | Significant | Family 110 |
| | | | down-regulation | member C with |
| | | | | sequence |
| | | | | similarity |
| SERPINB2 | ENSG00000197632 | −1.3377 | Significant | Serine protease |
| | | | down-regulation | inhibitor peptidase |
| | | | | inhibitor, clade B |
| | | | | (ovalbumin), |
| | | | | member 2 |
| HAS3 | ENSG00000103044 | −1.3738 | Significant | Hyaluronan |
| | | | down-regulation | synthase3 |
| PLAT | ENSG00000104368 | −1.552 | Significant | Plasminogen |
| | | | down-regulation | activator |
| ESM1 | ENSG00000164283 | −2.7641 | Significant | Endothelial cell |
| | | | down-regulation | specific molecule 1 |

### Example 6: Extraction of Cholesterol and Squalene in Epidermal Tissue Models Treated by PM2.5

In order to confirm the effect of PMs on the synthesis of skin cholesterol, a 3D epidermal tissue model (Epikutis PM1011, Biocell, Guangdong, China) was cultured in EpiGrowth medium (PY1021, Biocell, Guangdong, China) to study the effect on synthesis under the condition of 37° C. and 5% CO₂.

After 4 days of development, 3D epidermal tissues were cultured for 2, 4, 6 days in medium containing PM2.5 (50 μg/mL) or GTE (0.6%)+PM2.5 (50 μg/mL), without any treatment as a control. The total volume of the medium was 0.9 mL/well and the medium was changed daily. 3D-ETM samples were collected at different time points (2, 4, 6 days), the medium was decanted and the excess medium was carefully wiped off from the surface of the epidermal tissue and then stored at −20° C., three replicates for each condition. The 3D-ETS was carefully separated from the side of the culture well using a spatula, and then the sample was digested with 100 μL of Proteinase K (Ambion) at 55° C. for 30 minutes. The samples cultured under the same conditions were combined, and sonicated in an organic solvent (chloroform:methanol=2:1) in an ice water bath to extract lipid components. Then, samples were dried under nitrogen and stored at −80° C., and the lipid content was analyzed by LC-MS.

For LC-MS, the liquid chromatograph was equipped with C18, 1.8 μM, 100×2.1 mm columns, and the lipid samples was re-dissolved in the mobile phase at an injection volume of 1 μL. In this system, a 1100 binary pump was connected to two mobile phases: A. Acetonitrile:isopropanol=1:9, v/v, 0.1% formic acid; B. water:acetonitrile=4:6, v/v, 0.1% formic acid at a flow rate of 0.5 ml/min. The mobile phase continuous procedure was as follows: the phase B continued for 7 min from the linear gradient 99% to 50%, and phase B continued for 3 min from the linear gradient 50% to 1%, phase B is ocratically eluted at 1% for 3 minutes, and finally, phase B was isocratically eluted at 99% for 3 minutes, to equilibrate the columns. The parameters of Orbitrap MS were as follows: positive mode, spray voltage=4000V, gas pressure 1=30 psi, gas pressure 2=10 psi, scan range=150-1000 m/z. Analysis was performed using Progenesis QI.

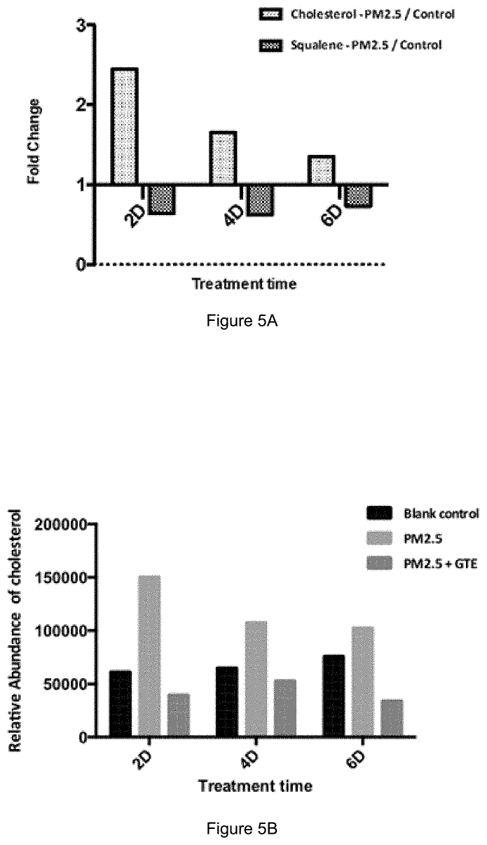

The results were shown in FIG. 5A and FIG. 5B. The transcriptomic results were validated using a 3D skin model. By simulating the changes in human skin under PM2.5 stimulation, the levels of cholesterol and intermediate squalene in the skin were measured at different treatment times. For example, FIG. 5A indicated that, after 2 days of stimulation with PM2.5, the cholesterol level in the skin reached the highest value, which was about 2.3 times higher than that of the control group. After that, the cholesterol level was dropped, and basically similar to the control group on the 6^{th} day. The cholesterol precursor showed a downward trend over time. This further demonstrated that, as the expression of some genes in the cholesterol metabolism pathway was significantly up-regulated, affecting the cholesterol metabolism pathway, the cholesterol level was eventually increased.

At the same time, the addition of GTE in the 3D skin models treated with PM2.5 could significantly inhibit the increase in cholesterol levels. For example, as shown in FIG. 5B, the cholesterol level remained basically unchanged in the blank control, but under the action of PM2.5, it was significantly higher than that of the control, and on the second day, it increased most, and then decreased. When PM2.5 and GTE were used simultaneously, the cholesterol level could be significantly inhibited at a normal level, or at least close to the normal level of the control. This fully demonstrated that the cholesterol level can be used to reflect that polyphenols can effectively improve and reverse the adverse effects of PM2.5 on the skin.

The above experimental results showed that PM2.5 induced the expressions of enzymes involved in the cholesterol metabolism process after contact with the skin, and caused a significant increase in cholesterol level in the skin on the second day, while GTE could effectively suppress this trend, regardless of the second day or sixth day after treatment, there was no significant fluctuation in the cholesterol level in the skin tissues in the GTE treatment group. This further suggested that polyphenols could maintain its normal level under the adverse effects of PM2.5, which sufficiently demonstrated polyphenols had significant improvement or reversal effect on the adverse effects of PMs on the skin SC. One skilled in the art can readily appreciate that polyphenols can protect skin SC from the harmful effects of PM2.5, thereby leaving the skin under protection.

In addition, squalene, as an important intermediate in the cholesterol metabolism process, has also been shown to be an important marker of environmental pollution levels in the skin. Therefore, we also tested the content of squalene under PM2.5 treatment in the 3D skin models. The results in FIG. 5A showed that, under the stimulation of PM2.5, the changing trend of squalene was opposite to that of cholesterol, which was lower than that of the control group. The possible reason may be that the squalene is a direct precursor of cholesterol, when the cholesterol level increases, the level of squalene is relatively decreased.

The invention shown and described herein may be implemented in the absence of any elements, limitations specifically disclosed herein. The terms and expressions used herein are for illustration rather than limitation, which do not exclude any equivalents of the features and portions described herein in the use of these terms and expressions, in addition, it should be understood that various modifications are feasible within the scope of the present invention. It is therefore to be understood that, although the invention has been particularly disclosed by various embodiments and alternative features, modifications and variations of the concepts described herein may be employed by those of skilled in the art, and such modifications and variations will fall into the scope of protection of the present invention as defined by the appended claims.

The contents of the articles, patents, patent applications, and all other documents and electronic information available or documented herein are incorporated herein by reference in their entirety, as if each individual publication is specifically and individually indicated for reference. The applicant reserves the right to incorporate any and all materials and information from any such article, patent, patent application or other document into this application.

### REFERENCES

Kerkhoff C, Benedyk M, Sopalla C, et al. HaCaT keratinocytes overexpressing the two 5100 proteins S100A8 and S100A9 showed an increased NADPH oxidase activity in response to elevated calcium levels[J]. 2006.
Cheng C H, Leferovich J, Zhang X M, et al. Keratin gene expression profiles after digit amputation in C57BL/6 vs. regenerative MRL mice imply an early regenerative keratinocyte activated-like state. [J]. Physiological Genomics, 2013, 45(11):409-21.
Sengupta A, Lichti U F, Carlson B A, et al. Targeted disruption of glutathione peroxidase 4 in mouse skin epithelial cells impairs postnatal hair follicle morphogenesis that is partially rescued through inhibition of COX-2[J]. Journal of Investigative Dermatology, 2013, 133(7):1731-1741.
Tassi E, McDonnell K, Gibby K A, et al. Impact of fibroblast growth factor-binding protein-1 expression on angiogenesis and wound healing[J]. American Journal of Pathology, 2011, 179(5):2220-2232.
Kastwoelbern H R, Dana S L, Cesario R M, et al. Rosiglitazone induction of Insig-1 in white adipose tissue reveals a novel interplay of peroxisome proliferator-activated receptor gamma and sterol regulatory element-binding protein in the regulation of adipogenesis. [J]. Journal of Biological Chemistry, 2004, 279(23):23908-23915.

1. A method for repairing damage to skin comprising the step of contacting one of polyphenolic substances with the skin, and wherein the damage is caused by particulate matter (PM).

2. The method according to claim 1, wherein the polyphenolic substance comes from plant products, microbial fermentation products or mammal products.

3. The method according to claim 2, wherein the plant is Camellia sinensis.

4. The method according to claim 1, wherein the polyphenol substance comprising Camellia sinensis polyphenols.

5. The method according to claim 1, the particulate matter is PM2.5.

6. The method according to claim 1, wherein the damage to the skin caused by particulate matter comprising the effects of the PM on the stratum corneum (SC).

7. The method according to claim 6, wherein the effects of the PM on the stratum corneum comprising effect or changing of cholesterol metabolism-related substance in the SC.

8. The method according to claim 7, wherein the cholesterol metabolism-related substances comprising one or two genes involved in the regulation of cholesterol metabolism.

9. The method according to claim 7, wherein the cholesterol metabolism-related substances comprising one or two enzymes involved in cholesterol metabolism.

10. The method according to claim 7, wherein the cholesterol metabolism-related substances comprising some of substrates or precursors of cholesterol in the cholesterol synthesis steps.

11. The method according to claim 8, wherein, the gene is selected from the group consisting of ACLY, ACSS2, HMGCR, HMGCS1, MVD, MSMD1, FDFT1, SQLE, LSS, LDLR, SCD, FASN, and INSIGL.

12. The method according to claim 11, wherein the gene is HMGCS1, LDLR or FASN.

13. The method according to claim 9, wherein, the enzyme is selected from the group consisting of ecarboxylase, Methylsterol Monooxygenase 1, Methylsterol Monooxygenase 1, Farnesyl-Diphosphate Farnesyltransferase 1, Squalene Epoxidase, Lanosterol Synthase, Low Density Lipoprotein Receptor, Stearoyl-CoA Desaturase, Fatty Acid Synthase.

14. The method according to claim 10, wherein the substrates or precursors are selected from the group consisting of acetate, citrate, acetyl-CoA, acetoacetyl-CoA, acetoacetyl-CoA, β-Hydroxy β-methylglutaryl-CoA, mevalonate, mevalonic acid-5-P, mevalonate-5-pyrophosphate, dimethylallyl pyrophosphate, farnesyl pyrophosphate, squalene, squalene-2,3epoxide, lanosterol.

15. The method according to claim 7, wherein the cholesterol metabolism-related substance is cholesterol.

16. The method according to claim 1, wherein the PMs are the PMs in the atmosphere.

17. A method for repairing the damage of skin's SC comprising the step of contacting polyphenols with the skin, wherein the SC has been damaged by PM.

18. The method according to claim 17, the PM is PM2.5.

19. The method according to claim 17, wherein, the damage to the SC is changing the metabolites of cholesterol metabolism in the SC.

20. The method according to claim 19, wherein the metabolites of cholesterol metabolism in the SC is cholesterol.
